(19) United States
(12) Patent Application Publication
(10) Pub. No.: US 2011/0014723 A1
(43) Pub. Date: Jan. 20, 2011

(54) COMPOSITIONS AND PROCESSES RELATING TO HUMAN BOCAVIRUS

(76) Inventors: Dean D. Erdman, Decatur, GA (US); Teresa Peret, Atlanta, GA (US)

Correspondence Address: GIFFORD,KRASS,SPRINKLE,ANDERSON&CITKOWSI, PC, 2701 W. BIG BEAVER ROAD, P.O. BOX 7021, SUITE 330, TROY, MI, 48007-7021, US

(21) Appl. No.: 12/922,364

(22) PCT Filed: Mar. 13, 2009
(86) PCT No.: PCT/US09/37142

§ 371 Date: Sep. 13, 2010

Related U.S. Application Data
(60) Provisional application No. 61/069,470, filed on Mar. 14, 2008.

(51) Int. Cl. G01N 33/53; C12P 21/00; C07K 14/005

(52) U.S. Cl. 436/501; 435/69.1; 530/350

(57) ABSTRACT

Non-replicating, antigenic, human bocavirus virus-like particles (HBoV VLPs) are provided by the present invention along with assays using the HBoV VLPs to detect anti-HBoV antibodies in a biological sample. Pharmaceutical compositions including HBoV VLPs and/or anti-HBoV antibodies are described herein along with novel antibodies generated using HBoV VLPs as an antigen. A recombinant baculovirus is provided including a DNA sequence encoding an expressible human bocavirus VP2 with or without a DNA sequence encoding an expressible human bocavirus VP1 polypeptide, and/or a non-HBoV peptide or protein, and culturing the cells to form the VP1 and/or VP2 proteins that self assemble to form the HBoV VLPs which are then amenable to isolation.

### REFERENCE TO RELATED APPLICATION

This application claims priority from U.S. Provisional Patent Application Ser. No. 61/069,470, filed Mar. 14, 2008, the entire content of which is incorporated herein by reference.

### FIELD OF THE INVENTION

This invention relates generally to compositions and processes relating to human bocavirus (HBoV). In specific embodiments, the instant invention relates to detection of antibodies specific for HBoV in a biological sample. Processes are described for rapid and sensitive detection of HBoV and/or HBoV antibodies in human and animal biological samples and quantification thereof. Diagnostic kits are provided for detection of HBoV and/or HBoV antibodies in a clinical, laboratory, or field setting. Compositions including HBoV antigens are provided in specific embodiments which stimulate production of HBoV specific antibodies. Further specific embodiments of the present invention relate to antibodies specific for HBoV.

### BACKGROUND OF THE INVENTION

Parvovirus designates a genus of the virus family Parvoviridae. The Parvovirus genus includes a number of small DNA viruses with icosaedric symmetry that require co-infection with another virus, usually an adenovirus (adeno-associated virus) or can replicate in the absence of a helper virus (autonomous parvovirus). Parvoviruses are capable of systemic infection of humans and other animals. Parvoviruses require proliferating host cells in order to replicate, so infection of respiratory and gut epithelium, hematopoietic cells, and transplacental infection of fetuses are frequent characteristics of parvoviruses and are associated with fetal infection and spontaneous abortion. Previously known human parvoviruses are the parvovirus B19, including genotypes A6 and V9 (genus Erythrovirus), and the presumably apathogenic adeno-associated viruses (genus Dependovirus). More recently, human parvovirus 4 and 5 have been identified in blood and liver of most immunocompromised patients but their clinical significance is so far unknown. Human bocavirus is a newly recognized parvovirus, tentatively placed in the genus Bocavirus along with a bovine and canine parvovirus, has been discovered in respiratory specimens from Swedish children with lower respiratory tract infection (Allander et al. PNAS 102:12891-12896 http://jcm.asm.org/cgi/ijlink?linkType=ABST&journalCode=pnas&resid=102/36/12891) and subsequently in children worldwide. These studies identified HBoV in 1.5 to 5.7% of children with respiratory disease suggesting a causal link between HBoV and respiratory disease. (Allander et al. PNAS 102:12891-12896; Ma, et al. J. Clin. Microbiol. 44:1132-1134; Sloots et al. J. Clin. Virol. 35:99-102).

The DNA of HBoV codes for two structural capsid proteins, VP1 and VP2, and two regulatory non-structural proteins, NS-1 and NS-2. As known for other parvoviruses, NS-1 and NS-2 are phosphorylated and localize to the nucleus and the cytoplasm, respectively. NS-1 serves to regulate viral DNA replication and participates viral gene expression. Particularly, NS-1 transactivates the promoter P38 and exhibits DNA binding, helicase and DNA nicking activities. Furthermore, NS-1 induces cytotoxic and/or cytostatic stress in sensitive host cells.

Conventional and real-time PCR assays have identified HBoV in respiratory specimens. Studies have pointed out an association of HBoV with asthma, wheezing, and hospitalized patients with pneumonia. Moreover, co-infection of HBoV with human respiratory syncytial virus, influenza A or B, human metapneumovirus, HPIV1-3, adenovirus and human coronavirus has been observed. More recently, HBoV has also been identified in stools of children with and without respiratory disease. However, the high rate of co-infection with other respiratory agents has posed a challenge to link HBoV to respiratory infections. Serological assays, in addition to evaluating the antibody acquisition to HBoV, may be important to elucidate the role of HBoV as a sole or co-infecting agent in respiratory diseases and to diagnose HBoV infection. Thus, there is a need for improved processes and reagents for detection and quantification of HBoV antibodies in biological samples.

### SUMMARY OF THE INVENTION

A process of producing non-replicating, antigenic, HBoV virus-like particles is provided by the present invention which includes introducing into a host cell a first recombinant expression vector including a DNA sequence encoding at least one structural protein of HBoV capsid. The host cell is cultured under conditions such that the structural protein is produced, and the protein then self assembles to form HBoV virus-like particles defining an internal space. The internal space contains no intact HBoV genome. The HBoV virus-like particles are isolated from the host cells for various uses.

The term “isolated” refers to materials separated from substances with which they are produced or naturally occur. The term “isolated” does not implicate absolute purity.

In preferred embodiments, the least one structural protein of HBoV capsid is HBoV VP2. Optionally, both HBoV capsid proteins VP1 and VP2 are encoded by the DNA sequence. In a further option, a second recombinant expression vector containing a DNA sequence encoding at least HBoV VP1 is introduced into the host cell.

In preferred processes of the invention, the recombinant expression vector is a baculovirus.

A non-replicating, antigenic, HBoV virus-like particle including at least one structural protein of HBoV capsid is provided according to embodiments of the present invention. In preferred embodiments, the non-replicating, antigenic, HBoV virus-like particles of the present invention include HBoV VP2 and are substantially free of HBoV VP1. In embodiments of inventive HBoV VLPs, the ratio of VP1:VP2 is higher compared to naturally occurring HBoV capsids. For example, optionally, the ratio of VP1:VP2 in HBoV VLPs of the present invention is in the range of 0.2:1-100:1, inclusive. In preferred embodiments, the ratio of VP1:VP2 is in the range of 0.25:1-1:1, inclusive.

A non-replicating, antigenic, HBoV virus-like particle is described according to embodiments of the present invention which includes an HBoV structural protein selected from VP1 and VP2 bonded to a non-HBoV protein.

A process for detection of an HBoV antibody in a biological sample is provided which includes contacting a first biological sample with a plurality of non-replicating, antigenic, HBoV virus-like particles and detecting the formation of a complex between an anti-HBoV antibody present in the first biological sample and the plurality of HBoV virus-like particles. A first signal is obtained which is indicative of the presence of an anti-HBoV antibody.

In further embodiments of an inventive process, the first biological sample is obtained from a subject in an acute phase of a viral disease. A second sample is obtained from the subject in a convalescent phase of a viral disease and the second biological sample is contacted with a plurality of isolated non-replicating, antigenic, HBoV virus-like particles. The formation of a complex between an anti-HBoV antibody present in the second biological sample and the HBoV virus-like particles is detected to obtain a second signal indicative of the presence of an anti-HBoV antibody. The first signal and second signal are compared to detect a different amount of an anti-HBoV antibody present in the second biological sample compared to the first biological sample. Where a greater amount of an anti-HBoV antibody is present in the second biological sample compared to the first biological sample, HBoV-associated disease is diagnosed.

A further method for detection of an HBoV antibody in a biological is provided according to the present invention wherein the detected anti-HBoV antibody is an IgM anti-HBoV antibody in a sample obtained from a subject, indicative of a current or recent HBoV infection.

An anti-HBoV vaccine is provided by embodiments of the present invention which includes non-replicating, antigenic, HBoV virus-like particles admixed with a pharmaceutically acceptable carrier.

A process of delivering a cargo moiety to a cell is described according to the present invention which includes introducing a cargo moiety into an internal space defined by a non-replicating, antigenic, HBoV virus-like particle and contacting the HBoV virus-like particle and a cell. Exemplary cargo moieties include a label, an antigen, a nucleic acid sequence encoding a protein or peptide, and/or a therapeutic agent.

An anti-HBoV antibody assay kit which includes isolated non-replicating, antigenic, HBoV virus-like particles and at least one ancillary reagent.

A recombinant baculovirus is detailed according to the present invention which includes a DNA sequence encoding HBoV VP1 and/or VP2. In particular embodiments, a recombinant baculovirus is provided by the present invention which includes a DNA sequence encoding the protein of SEQ ID No. 1 or a variant thereof.

An antibody which specifically binds to HBoV and which does not specifically bind to parvovirus B19 is described according to the present invention.

An assay for HBoV is provided which includes contacting a biological sample and an antibody specific for HBoV. A complex formed by HBoV in the biological sample and the antibody is detected in the assay. An inventive kit described herein for assay for HBoV in a sample includes an antibody specific for HBoV and at least one an ancillary reagent.

A process of producing HBoV capsids is provided that includes introducing into a host cell a recombinant DNA molecule containing an expression vector and a DNA sequence encoding a structural HBoV polypeptide, with the proviso that one or more nucleic acid sequences encoding nonstructural HBoV polypeptides are not included in the DNA sequence. The host cells are cultured the under conditions such that said structural proteins are produced and self assemble to form the capsids which are then amenable to isolation. The host cells containing the recombinant DNA molecule are optionally in culture or in vivo in a non-human subject.

In examples described herein, insect host cells containing the recombinant DNA molecule are cultured to support the growth of a recombinant baculovirus containing the gene coding for the HBoV major capsid protein (VP2). The expressed HBoV VP2 assembles to form “empty” capsids which are virus-like particles (VLPs).

Antibodies are described herein that recognize HBoV capsid virus-like particles (VLPs).

An HBoV antigen is also provided that includes purified HBoV capsid with a minor structural protein to major structural protein ratio higher than the protein ratio of the naturally occurring HBoV capsid. An HBoV antigen is also provided that is essentially a purified HBoV capsid of major structural proteins free of minor structural proteins.

A diagnostic assay process for detection of HBoV infection is provided that includes contacting a sample from a patient suspected of being infected with HBoV with an HBoV antigen and then detecting the formation of a complex between anti-HBoV antibodies present in the sample and the HBoV antigen introduced. An anti-HBoV vaccine is provided inclusive of HBoV antigen and a pharmaceutically acceptable carrier.

A process of packaging and transferring genetic information is provided that includes introducing a recombinant DNA molecule containing an expression vector for expression of a heterologous peptide or protein into an HBoV VLP; and introducing the VLP into a host cell to express the protein.

A diagnostic kit is provided including HBoV antigen, particularly HBoV VLPs, as described herein with ancillary reagents producing a discernable change when an anti-HBoV antigen antibody is present in a sample collected from an individual suspected of being infected or having been infected with HBoV. The diagnostic kit optionally includes a discernable signal-producing system.

A recombinant baculovirus is provided including a DNA segment encoding a minor or a major structural polypeptide of an HBoV. Autographa california nuclear polyhedrosis virus represents a preferred recombinant baculovirus.

A fusion protein presenting HBoV VLP is provided including a major structural HBoV protein and a non-unique region of a minor structural HBoV protein joined to a non-HBoV protein.

Enzyme linked immunoadsorbant assays are provided by embodiments of the present invention that include capture of HBoV antibodies present in a sample, such as a sample obtained from a patient suspected of being infected or having been infected with HBoV with a corresponding HBoV capsid antigen, particularly HBoV VLPs.

### BRIEF DESCRIPTION OF THE DRAWINGS

FIG. 1 is a reproduction of an electron micrograph of HBoV virus-like particles obtained after Sf9 cells transfection with recombinant baculovirus containing the HBoV VP2 gene (72 hours p.i.);

FIG. 2 is a reproduction of an electron micrograph of HBoV virus-like particles obtained after High5 cell infection (mid-scale production) with recombinant baculovirus containing the HBoV VP2 gene (96 hours p.i.);

FIG. 3A is a reproduction of a photograph showing results of an indirect immunofluorescence assay (IFA) using human sera from patients positive for HBoV incubated with SF-9 cells infected with recombinant baculovirus-expressed HBoV protein (VP2) (72 hours p.i.);

FIG. 3B is a reproduction of a photograph showing results of an indirect immunofluorescence assay (IFA) using human sera from patients positive for HBoV incubated with uninfected Sf9 cells (72 hours p.i.);

FIG. 4A is a reproduction of a photograph showing results of an IFA using sera from mice immunized with purified HBoV VLPs incubated with SF-9 cells infected with recombinant baculovirus-expressed HBoV protein (VP2) (72 hours p.i.);

FIG. 4B is a reproduction of a photograph showing results of an IFA using sera from mice immunized with purified HBoV VLPs incubated with SF-9 cells infected with recombinant baculovirus-expressed HBoV protein (VP2) (72 hours p.i.);

FIG. 4C is a reproduction of a photograph showing results of an IFA using sera from mice immunized with purified HBoV VLPs incubated with uninfected Sf9 cells (72 hours p.i.);

FIG. 5A is a reproduction of a photograph showing results of an IFA using a monoclonal antibody to HBoV VLPs incubated with Sf9 cells infected with recombinant baculovirus-expressed HBoV protein (VP2) (72 hours p.i.);

FIG. 5B is a reproduction of a photograph showing results of an IFA using a monoclonal antibody to HBoV VLPs incubated with uninfected Sf9 cells;

FIG. 6 is a graph showing results of IgM/IgG enzyme immunoassays (EIA) of eighty-one sera from healthy adult blood donors and infants incubated with HBoV VLP and indicating that all sera are positive except one sample from an infant (cross-hatched dot), and a B19 IgM IgG negative control serum also tested positive for HBoV antibodies (unfilled dot);

FIG. 7A is a reproduction of an electron micrograph showing results of immunoassay using HBoV positive serum incubated with HBoV VLPs, where binding is detected by an anti-human secondary antibody conjugated with gold particles (dark spots) indicating specific binding of the serum antibodies to the VLPs;

FIG. 7B is a reproduction of an electron micrograph showing results of immunoassay using HBoV positive serum incubated with HBoV VLPs, where binding is detected by an anti-human secondary antibody conjugated with gold particles (dark spots) indicating specific binding of the serum antibodies to the VLPs; and

FIG. 7C is a reproduction of an electron micrograph showing results of immunoassay using HBoV negative serum incubated with HBoV VLPs and detected by an anti-human secondary antibody conjugated with gold particles.

### DETAILED DESCRIPTION OF THE PREFERRED EMBODIMENTS

The recent identification of HBoV infection in children with lower respiratory tract infection suggests a causal relationship between viral presence and onset of disease. The instant invention has numerous uses including, but not limited to, detection of HBoV antibodies in biological samples, diagnosis of HBoV infection, identification of individuals previously or currently infected with HBoV, as an antigen for generation of antibodies and for the development of therapeutics for prophylaxis or treatment of disease associated with HBoV infection.

Particular techniques may be used in accordance with the present invention which are conventional techniques of molecular biology, cell biology, recombinant nucleic acids, immunology and the like. Such techniques are described in detail in standard texts such as E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; B. K. C. Lo, Antibody Engineering: Methods and Protocols, Methods in Molecular Biology, Humana Press, 2003; F. M. Ausubel et al., Eds., Short Protocols in Molecular Biology, Current Protocols, Wiley, 2002; Crowther, J. R., The ELISA Guidebook (Methods in Molecular Biology), Humana Press, 2000; Wild, D., The Immunoassay Handbook, 3rd Edition, Elsevier Science, 2005; and J. Sambrook and D. W. Russell, Molecular Cloning: A Laboratory Manual, Cold Spring Harbor Laboratory Press, 3rd Ed., 2001.

As used herein, the terms “antibody” and “antibodies” relate to monoclonal antibodies, polyclonal antibodies, bispecific antibodies, multispecific antibodies, human antibodies, humanized antibodies, chimeric antibodies, camelized antibodies, single domain antibodies, single-chain Fvs (scFv), single chain antibodies, disulfide-linked Fvs (sdFv), and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site. Immunoglobulin molecules are of any type (e.g., IgG, IgE, IgM, IgD, IgA and IgY), class (e.g., IgG1, IgG2, IgG3, IgG4, IgA1 and IgA2), or subclass.

As used herein, the term “antibody fragment” defines a fragment of an antibody that immunospecifically binds to an HBoV virus, any epitope of the HBoV virus or HBoV VLP. Antibody fragments may be generated by any technique known to one of skill in the art. For example, Fab and F(ab′)₂ fragments may be produced by proteolytic cleavage of immunoglobulin molecules, using enzymes such as papain (to produce Fab fragments) or pepsin (to produce F(ab′) 2 fragments). F(ab′) 2 fragments contain the complete light chain, and the variable region, the CH 1 region and the hinge region of the heavy chain. Antibody fragments are also produced by recombinant DNA technologies. Antibody fragments may be one or more complementarity determining regions (CDRs) of antibodies.

HBoV virus-like particles (VLPs) are provided according to the present invention. The term “virus-like particle” refers to a capsid defining an internal space. The internal space defined by the capsid is “empty” of an intact HBoV genome and the HBoV VLPs of the present invention are therefore non-replicating and incapable of causing HBoV-associated disease.

Naturally occurring HBoV capsids includes two structural capsid proteins, VP1 and VP2. VLPs of the present invention are compositionally distinct from naturally occurring HBoV capsids, containing a different ratio of VP1:VP2 compared to naturally occurring HBoV capsids. Based on analogy to parvovirus B19, it is believed that naturally occurring HBoV capsids contain about 90-95% by weight VP2 protein and about 5-10% VP1 In preferred embodiments, VLPs of the present invention contain about 95-100% by weight VP2 protein and are substantially free of VP1 protein. For example, VLPs substantially free of VP1 protein contain 0.1% or less VP1 protein by weight of the VLP. In embodiments of inventive HBoV VLPs, the ratio of VP1:VP2 is higher compared to naturally occurring HBoV capsids. For example, optionally, the ratio of VP1:VP2 in HBoV VLPs of the present invention is in the range of 0.2:1-100:1, inclusive. In preferred embodiments, the ratio of VP1:VP2 is in the range of 0.25:1-1:1, inclusive.

Genes encoding HBoV structural proteins VP1 and VP2 and non-structural proteins have been identified and sequenced from various sources worldwide. Structural proteins VP1 and VP2 from different bocavirus strains are highly homologous, having a high degree of similarity between any two VP1 proteins or any two VP2 proteins as described in T. Chieochansin et al. Virus Research, 129:54-57, 2007.

HBoV VLPs include any HBoV VP1 protein and/or any HBoV VP2 protein. A particular HBoV VP2 protein is disclosed herein as SEQ ID No. 1. Additional HBoV VP2 proteins include those identified by Qu, X. W. et al. having GenBank Accession No. ABE73069; Lu, X. D. et al having GenBank Accession No. ABY55264; Chieochansin, T. et al. having GenBank Accession No. ABX57870; and Chieochansin, T. et al. having GenBank Accession No. ABX57866. HBoV VP1 proteins include HBoV VP1 protein disclosed herein as SEQ ID No. 5; human VP1 identified by Qu, X. W. et al. having GenBank Accession No. ABF50818; Qu, X. W. et al. having GenBank Accession No. ABF50816; Qu, X. W. et al. having GenBank Accession No. ABE73071; Chieochansin, T. et al. having GenBank Accession No. ABX57869; and Chieochansin, T. et al. having GenBank Accession No. ABX57865.

In addition to these VP1 and VP2 amino acid sequences, the terms VP1 and VP2 amino acid sequences encompass variants of VP1 and VP2 which may be included in HBoV VLPs of the present invention. As used herein, the term “variant” defines either a naturally occurring genetic mutant of the HBoV virus or a recombinantly prepared variation of the HBoV virus, each of which contain one or more mutations in its genome compared to the HBoV virus of strain st1, GenBank Accession no. DQ00495. The term “variant” may also refer to either a naturally occurring variation of a given peptide or a recombinantly prepared variation of a given peptide or protein in which one or more amino acid residues have been modified by amino acid substitution, addition, or deletion.

Highly preferred are HBoV VP1 and VP2 proteins having at least 90%, 91%, 92%, 93%, 94%, 95%, 96%, 97%, 98% or 99% identity to SEQ ID No. 1 or SEQ ID No.5.

Mutations can be introduced using standard molecular biology techniques, such as site-directed mutagenesis and PCR-mediated mutagenesis. One of skill in the art will recognize that one or more amino acid mutations can be introduced without altering the functional properties of VP1 or VP2 proteins. For example, one or more amino acid substitutions, additions, or deletions can be made without altering the functional properties of HBoV proteins.

Conservative amino acid substitutions can be made in VP1 or VP2 proteins to produce VP1 or VP2 protein variants. Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

HBoV VP1 or VP2 variants can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non limiting example of a mathematical algorithm utilized for the comparison of two sequences is the algorithm of Karlin and Altschul, 1990, PNAS 87:2264 2268, modified as in Karlin and Altschul, 1993, PNAS. 90:5873 5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, wordlength=12 to obtain nucleotide sequences homologous to a nucleic acid molecules of the present invention. BLAST protein searches are performed with the XBLAST program parameters set, e.g., to score 50, wordlength=3 to obtain amino acid sequences homologous to a protein molecule of the present invention. To obtain gapped alignments for comparison purposes, Gapped BLAST are utilized as described in Altschul et al., 1997, Nucleic Acids Res. 25:3389 3402. Alternatively, PSI BLAST is used to perform an iterated search which detects distant relationships between molecules (Id.). When utilizing BLAST, Gapped BLAST, and PSI Blast programs, the default parameters of the respective programs (e.g., of XBLAST and NBLAST) are used (see, e.g., the NCBI website). Another preferred, non limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

VLPs are produced using recombinant nucleic acid technology. VLP production includes introducing a recombinant expression vector encompassing a DNA sequence encoding one or more structural proteins, VP1 and/or VP2 of HBoV capsids into a host cell. The DNA sequence is optionally exclusive of genes encoding non-structural proteins.

Specific nucleic acid sequences encoding HBoV VP1 and/or VP2 introduced into a host cell to produce HBoV VLPs are those encoding SEQ ID No. 1 and those encoding the HBoV VP2 proteins identified by Qu, X. W. et al. having GenBank Accession No. ABE73069; Lu, X. D. et al having GenBank Accession No. ABY55264; Chieochansin, T. et al. having GenBank Accession No. ABX57870; and Chieochansin, T. et al. having GenBank Accession No. ABX57866 and/or the HBoV VP1 proteins identified by Qu, X. W. et al. having GenBank Accession No. ABF50818; Qu, X. W. et al. having GenBank Accession No. ABF50816; Qu, X. W. et al. having GenBank Accession No. ABE73071; Chieochansin, T. et al. having GenBank Accession No. ABX57869; and Chieochansin, T. et al. having GenBank Accession No. ABX57865; and variants of these. A specific DNA sequence encoding VP2 is set forth as SEQ ID No. 2. A specific DNA sequence encoding VP1 is set forth as SEQ ID No. 6.

It is appreciated that due to the degenerate nature of the genetic code, alternate nucleic acid sequences encode HBoV VP1, VP2 and variants thereof, and that such alternate nucleic acids may be included in an expression vector and expressed to produce HBoV VLPs of the present invention.

In embodiments of the present invention, a nucleic acid sequence which is substantially identical to SEQ ID No. 2 is included in an expression vector and expressed to produce HBoV VLPs of the present invention. In further embodiments of the present invention, a nucleic acid sequence which is substantially identical to SEQ ID No. 6 is included in an expression vector and expressed to produce HBoV VLPs of the present invention.

A nucleic acid sequence which is substantially identical to SEQ ID No. 2 is characterized as having a complementary nucleic acid sequence capable of hybridizing to SEQ ID No. 2 under high stringency hybridization conditions. Similarly, a nucleic acid sequence which is substantially identical to SEQ ID No. 6 is characterized as having a complementary nucleic acid sequence capable of hybridizing to SEQ ID No. 6 under high stringency hybridization conditions.

The term “nucleic acid” refers to RNA or DNA molecules having more than one nucleotide in any form including single-stranded, double-stranded, oligonucleotide or polynucleotide. The term “nucleotide sequence” refers to the ordering of nucleotides in an oligonucleotide or polynucleotide in a single-stranded form of nucleic acid.

The term “complementary” refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′.

The terms “hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002.

An example of high stringency hybridization conditions is hybridization of nucleic acids over about 100 nucleotides in length in a solution containing 6×SSC, 5×Denhardt's solution, 30% formamide, and 100 micrograms/ml denatured salmon sperm at 37° C. overnight followed by washing in a solution of 0.1×SSC and 0.1% SDS at 60° C. for 15 minutes. SSC is 0.15M NaCl/0.015M Na citrate. Denhardt's solution is 0.02% bovine serum albumin/0.02% FICOLL/0.02% polyvinylpyrrolidone. Under highly stringent conditions, SEQ ID No. 2 or SEQ ID No. 6 will hybridize to the complement of substantially identical targets and not to unrelated sequences.

The term “expression vector” refers to a recombinant vehicle for introducing a DNA sequence encoding one or more structural proteins of HBoV capsids into a host cell where the DNA sequence is expressed to produce the one or more structural proteins. In particular embodiments, an expression vector including SEQ ID No. 2 or a substantially identical nucleic acid sequence is expressed to produce HBoV VP2 and self-assembled VLPs in cells containing the expression vector.

In additional embodiments, an expression vector including SEQ ID No. 6 or a substantially identical nucleic acid sequence is expressed to produce HBoV VP1 and self-assembled VLPs in cells containing the expression vector.

In further embodiments, an expression vector including SEQ ID No. 2 or a substantially identical nucleic acid sequence and SEQ ID No. 6 or a substantially identical nucleic acid sequence is expressed to produce HBoV VP2, HBoV VP1 and self-assembled VLPs in cells containing the expression vector.

In still further embodiments, a first expression vector including SEQ ID No. 2 or a substantially identical nucleic acid sequence and a second expression vector including SEQ ID No. 6 or a substantially identical nucleic acid sequence are both expressed to produce HBoV VP2, HBoV VP1 and self-assembled VLPs in cells containing the expression vectors.

In addition to one or more DNA sequences encoding one or more structural proteins of HBoV capsids, one or more DNA sequences encoding additional proteins can be included in an expression vector. For example, such additional proteins include non-HBoV proteins such as reporters, including, but not limited to, beta-galactosidase, green fluorescent protein and antibiotic resistance reporters; and antigens.

Expression vectors are known in the art and include plasmids and viruses, for example. An expression vector contains a DNA molecule that includes segment encoding a polypeptide of interest operably linked to one or more regulatory elements that provide for transcription of the segment encoding the polypeptide of interest. Such regulatory elements include, but are not limited to, promoters, terminators, enhancers, origins of replication and polyadenylation signals.

In particular embodiments, the recombinant expression vector encodes at least HBoV VP2 of SEQ ID No. 1, a protein having at least 95% identity to SEQ ID No. 1, a protein encoded by SEQ ID No. 2, or a protein encoded by a nucleic acid sequence substantially identical to SEQ ID No. 2.

Optionally, the recombinant expression vector encodes HBoV VP1 of SEQ ID No. 5, a protein having at least 95% identity to SEQ ID No. 5, a protein encoded by SEQ ID No. 6, or a protein encoded by a nucleic acid sequence substantially identical to SEQ ID No. 6.

In further embodiments, the recombinant expression vector encodes HBoV VP2 of SEQ ID No. 1, a protein having at least 95% identity to SEQ ID No. 1, a protein encoded by SEQ ID No. 2, or a protein encoded by a nucleic acid sequence substantially identical to SEQ ID No. 2; and HBoV VP1 of SEQ ID No. 5, a protein having at least 95% identity to SEQ ID No. 5, a protein encoded by SEQ ID No. 6, or a protein encoded by a nucleic acid sequence substantially identical to SEQ ID No. 6.

A preferred expression vector of the present invention is a baculovirus.

Expression of VP1 and/or VP2 encoded by a recombinant expression vector is accomplished by introduction of the expression vector into a eukaryotic or prokaryotic host cell expression system such as an insect cell, mammalian cell, yeast cell, bacterial cell or any other single or multicellular organism recognized in the art. In preferred embodiments, a eukaryotic host cell is used. Host cells are optionally primary cells or immortalized derivative cells. Immortalized cells are those which can be maintained in-vitro for at least 5 replication passages.

Host cells containing the recombinant expression vector are maintained under conditions wherein structural proteins of HBoV capsids are produced. The VP1 and/or VP2 capsid proteins self-associate to produce VLPs of the present invention in the host cell.

The invention provides a host cell containing a nucleic acid sequence according to the invention. Host cells may be cultured and maintained using known cell culture techniques such as described in Celis, Julio, ed., 1994, Cell Biology Laboratory Handbook, Academic Press, N.Y. Various culturing conditions for these cells, including media formulations with regard to specific nutrients, oxygen, tension, carbon dioxide and reduced serum levels, can be selected and optimized by one of skill in the art.

A preferred cell line of the present invention is a eukaryotic cell line, preferably an insect cell line, such as Sf9, transiently or stably expressing one or more full-length or partial HBoV proteins. Such cells can be made by transfection (proteins or nucleic acid vectors), infection (viral vectors) or transduction (viral vectors). The cell lines for use in the present invention are cloned using known cell culture techniques familiar to one skilled in the art. The cells are cultured and expanded from a single cell using commercially available culture media under known conditions suitable for propagating cells.

In a preferred embodiment HBoV VLPs are produced by infection of a host cell with a recombinant baculovirus. The recombinant baculovirus optionally encodes a minor structural protein, a major structural protein, or both.

It is appreciated that a single baculovirus may encode either a single structural protein or multiple structural proteins. In a preferred embodiment a single baculovirus encodes both major and minor structural proteins. In a more preferred embodiment, multiple recombinant baculovirus constructs are used each encoding a single or multiple structural proteins. In a most preferred embodiment, a first recombinant baculovirus with a DNA segment encoding a minor structural HBoV protein is coadministered to a host cell with a second recombinant baculovirus with a DNA segment encoding a major structural HBoV protein. The first and second recombinant baculoviruses optionally encode the same or different structural proteins. The resulting infected cells are then cultured under conditions whereby the encoded structural proteins from the respective recombinant baculoviruses are produced and self assemble to form the capsids. The resulting HBoV VLPs are then optionally and preferably isolated.

In further preferred embodiments, the recombinant baculovirus encodes at least HBoV VP2 of SEQ ID No. 1, a protein having at least 95% identity to SEQ ID No. 1, a protein encoded by SEQ ID No. 2, or a protein encoded by a nucleic acid sequence substantially identical to SEQ ID No. 2.

Optionally, the recombinant baculovirus encodes HBoV VP1 of SEQ ID No. 5, a protein having at least 95% identity to SEQ ID No. 5, a protein encoded by SEQ ID No. 6, or a protein encoded by a nucleic acid sequence substantially identical to SEQ ID No. 6.

In a further option, the recombinant baculovirus encodes HBoV VP2 of SEQ ID No. 1, a protein having at least 95% identity to SEQ ID No. 1, a protein encoded by SEQ ID No. 2, or a protein encoded by a nucleic acid sequence substantially identical to SEQ ID No. 2; and HBoV VP1 of SEQ ID No. 5, a protein having at least 95% identity to SEQ ID No. 5, a protein encoded by SEQ ID No. 6, or a protein encoded by a nucleic acid sequence substantially identical to SEQ ID No. 6.

Any suitable baculovirus known in the art is operable in the instant inventive process. Preferably, the baculovirus is Autographa california nuclear polyhedrosis virus.

Processes for infecting cells with baculovirus are known in the art. Following infection of a host cell the inventive process proceeds by culturing the host cells under conditions such that structural protein(s) is produced that thereby self assemble to form one or more capsids. The terms “capsid” and “VLP” are used interchangeably herein. The capsids are subsequently isolated by processes known in the art. The structural proteins encoded by the baculovirus are optionally major structural proteins or minor structural proteins. The major structural protein is optionally VP2 and the minor structural protein is optionally VP1.

A VLP of the present invention optionally includes a non-HBoV protein or peptide in contact with or bonded to at least one of the structural HBoV proteins VP1 and VP2. Bonding of the non-HBoV protein or peptide is achieved, for example, by expression of a fusion construct including a nucleic acid sequence encoding VP1 or VP2 and the non-HBoV protein or peptide. Thus, the non-HBoV protein or peptide is optionally a fusion protein or peptide wherein the non-HBoV protein is synthesized as a single polypeptide chain with an HBoV structural protein.

The non-HBoV protein is optionally fused with glutathione-S-transferase (GST) for rapid isolation. An HBoV protein is also optionally fused to GST.

Chemical bonding methods are optionally used to bond a VLP and a non-HBoV protein or peptide, illustratively including reaction using a cross-linking agent such as carbodiimide or glutaraldehyde.

In particular embodiments, the non-HBoV protein or peptide included in the VLP includes one or more antigenic epitopes such that the VLP serves to present the one or more antigenic epitopes to the immune system of a subject to induce antibody generation.

In a further option, the non-HBoV protein or peptide is a targeting moiety such as a receptor ligand or receptor. A targeting moiety is included in the VLP to direct the VLP to a target, such as to a particular cell type.

In one option, a recombinant baculovirus includes an expression vector encoding a non-HBoV protein. In a preferred embodiment a first recombinant baculovirus encoding VP2 is used to co-infect a host cell with a second recombinant baculovirus encoding VP1 and/or a non-HBoV protein. The infected host cells are then cultured under conditions known in the art to result in expression of the proteins. The expressed proteins then self assemble to form HBoV VLPs. The HBoV VLPs are optionally and preferably isolated from the host cells.

HBoV VLPs produced in a host cell are optionally isolated. The term “isolated” in reference to an HBoV VLP describes an HBoV VLP which is separated from a cell in which the HBoV VLP is produced and which is substantially free of host cell components not intended to be associated with the HBoV VLP. Generally, HBoV VLPs are separated from whole cell extracts of host cells. Numerous processes of isolating viral capsids are known in the art and are applicable to isolation of HBoV VLPs illustratively including sucrose continuous and discontinuous gradients, cesium chloride single and multi-density gradient centrifugation, size-exclusion chromatography, antigen capture chromatography, affinity chromatography, or other suitable process known in the art. An exemplary method for isolating HBoV VLPs of the present invention is described in Gillock, E T. et al, 1997. J. Virol., 71:2857-2865.

HBoV VLPs having different compositions, that is, different “types” of HBoV VLPs are optionally present in a composition of the present invention. For example, HBoV VLPs including HBoV VP2 and substantially free of VP1 are optionally included in a composition with antigen presenting HBoV VLPs including a non-HBoV protein or peptide and/or HBoV VLPs containing a cargo moiety.

### Detection of Anti-HBoV Antibodies

HBoV VLPs are used to detect anti-HBoV antibodies in a biological sample according to embodiments of a process of the present invention.

The term “biological sample” refers to a sample obtained from a biological organism, a tissue, cell, cell culture medium, or any medium suitable for mimicking biological conditions, or from the environment. Non-limiting examples include, saliva, gingival secretions, cerebrospinal fluid, gastrointestinal fluid, mucous, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, and vitreal fluid, and nasal secretions. In a preferred embodiment, the antigens are contained in serum, whole blood, nasopharyngeal fluid, other respiratory fluid.

A process of detecting anti-HBoV antibodies in a biological sample according to the present invention includes contacting a biological sample with recombinant HBoV VLPs and detecting formation of a complex between anti-HBoV antibodies present in the biological sample and the HBoV VLPs. Formation of the complex between anti-HBoV antibodies present in the biological sample and the HBoV VLPs is indicative of exposure of the subject to HBoV sufficient to activate the immune system of the subject to produce anti-HBoV antibodies. Formation of the complex specifically indicates presence of anti-HBoV antibodies since other respiratory virus antibodies, particularly parvovirus B19 antibodies, do not form a complex with the HBoV VLPs.

In a preferred embodiment, HBoV VLPs are used to detect anti-HBoV antibodies in a biological sample to diagnose current and recent HBoV infection in a subject. Diagnosis of HBoV infection according to embodiments of the present invention is achieved using at least two samples obtained from a subject, including at least one sample taken during acute disease and at least one sample taken during the convalescent phase, typically about 2 weeks apart. The samples are assayed for anti-HBoV antibodies. In a subject having a current HBoV infection a significant increase in anti-HBoV antibody titer, typically 4-fold or more, is observed in the sample taken during the convalescent phase compared to the sample taken during acute disease.

In a further embodiment of a diagnostic assay for current HBoV infection, HBoV VLPs are used to assay anti-HBoV IgM in a sample from a subject. Anti-HBoV IgM is produced during infection and decreases to undetectable levels following recovery from the infection. Recovery is indicated, for instance, by absence of respiratory symptoms and/or when HBoV DNA is not detectable when assayed by PCR in a sample obtained from a subject.

Assay for anti-HBoV IgM in a biological sample includes contacting HBoV VLPs with the biological sample obtained from a subject and detecting a complex formed between anti-HBoV IgM and HBoV VLPs. Detection is preferably achieved by contacting the complex with a labeled anti-IgM secondary antibody. Detection of anti-HBoV IgM in the biological sample is indicative of current and recent HBoV infection in the subject.

In a further preferred embodiment HBoV VLPs are used in a process of assessing the immune status of an individual with respect to past or present exposure to an HBoV antigen in HBoV infection susceptible organisms, particularly in a human subject.

A process of assessing the immune status of an individual according to the present invention includes contacting a biological sample with recombinant HBoV VLPs and detecting formation of a complex between anti-HBoV antibodies present in the biological sample and the HBoV VLPs. Formation of the complex between anti-HBoV antibodies present in the biological sample and the HBoV VLPs is indicative of exposure of the subject to HBoV sufficient to activate the immune system of the subject to produce anti-HBoV antibodies. Formation of the complex specifically indicates presence of anti-HBoV antibodies since other respiratory virus antibodies, particularly parvovirus B19 antibodies, do not form a complex with the HBoV VLPs.

The instant inventive processes are amenable to use in a subject, particularly a human subject, or other organism capable of infection by HBoV.

Detecting formation of a complex between anti-HBoV antibodies present in a biological sample and HBoV VLPs is achieved by any of various methods known in the art, illustratively including detection of a label attached to HBoV VLPs or attached to the anti-HBoV antibodies. The term “label” or “labeled” refers to any composition which can be used to detect, qualitatively or quantitatively, a substance attached to the label. Suitable labels include a fluorescent moiety, a radioisotope, a chromophore, a bioluminescent moiety, an enzyme, a magnetic particle, an electron dense particle, and the like. The term “label” or “labeled” is intended to encompass direct labeling of HBoV VLPs or an antibody by coupling (i.e., physically linking) a detectable substance to the HBoV VLPs or antibody, as well as indirect labeling of the HBoV VLPs or antibody by interaction with another reagent that is directly labeled. An example of indirect labeling of a primary antibody includes detection of a primary antibody using a fluorescently labeled secondary antibody.

Labels used in detection of complex formation depend on the detection process used. Such detection processes are incorporated in particular assay formats illustratively including ELISA, western blot, immunoprecipitation, immunocytochemistry, immuno-fluorescence assay, liquid chromatography, flow cytometry, other detection processes known in the art, or combinations thereof.

In a preferred embodiment an ELISA is used to detect the presence of HBoV antibodies in a biological sample.

In a preferred configuration of an ELISA for HBoV antibodies, HBoV VLPs are coated on a support such as a microtiter plate, beads, slide, silicon chip or other solid support such as a nitrocellulose or PVDF membrane. A biological sample is incubated with the HBoV VLPs on the support and the presence of complex between antibodies to HBoV and HBoV VLPs is detected by standard ELISA protocols. For example, a complex between HBoV VLPs and HBoV antibodies is detected by reaction of a labeled secondary antibody with the anti-HBoV antibodies and detection of the label.

Another example of an ELISA for HBoV antibodies is a sandwich ELISA. One embodiment of a sandwich ELISA includes depositing a binding antibody onto a solid support. The binding antibody is optionally a non-competing antibody that recognizes HBoV VLPs. The binding antibody is incubated with HBoV VLPs. The complex is washed to remove any unbound material and a detectable label, such as a fluorescently labeled antibody directed to HBoV VLPs, is applied. The detectable label is detected, if present, indicating the presence of anti-HBoV antibody in the biological sample.

Alternatively, the binding antibody deposited on the support is an antibody specific for human IgG or IgM. A biological sample is incubated with the binding antibody on the support to form a complex between the binding antibody and antibodies in the sample. Detectably labeled HBoV VLPs are incubated with the complex, binding to any HBoV antibodies captured by the binding antibody. Detection of the label indicates presence of the HBoV antibodies. Further details of ELISA assays in general are found in Crowther, J. R., The ELISA Guidebook (Methods in Molecular Biology), Humana Press, 2000; and Wild, D., The Immunoassay Handbook, 3rd Edition, Elsevier Science, 2005.

In a particular embodiment of a process of the present invention, an HBoV VLP-based serological assay is used to complement a PCR assay for the detection and measurement of the presence of HBoV in a biological sample. PCR detection of HBoV is described in detail in Lu, X. et al., Real-Time PCR Assays for Detection of Bocavirus in Human Specimens, J. of Clin. Micro., Vol. 44:3231-3235, 2006. The process of detecting HBoV antibodies in a, biological sample is optionally performed in parallel with the same or control biological samples that are used to detect HBoV gene sequences such as NP-1, NS1, or VP2.

An HBoV antibody detection kit is provided including one or more types of HBoV VLPs and ancillary reagents for use in detecting anti-HBoV antibodies in a biological sample. Ancillary reagents are any signal producing system materials for detection of a complex between an anti-HBoV antibody and an HBoV VLP in any suitable detection process such as ELISA, western blot, immunoprecipitation, immunocytochemistry, immuno-fluorescence, mass spectrometry, or other assay known in the art.

Optionally, an anti-human bocavirus antibody assay kit according to embodiments of the present invention includes HBoV VLPs attached to a solid substrate. Suitable solid substrates include, but are not limited to, microtiter plates, chips, tubes, membranes, such as nylon or nitrocellulose membranes, and particles, such as beads. Attachment of protein-containing materials to solid substrates is well-known in the art and includes, but is not limited to, adsorption.

In a preferred embodiment, an HBoV antibody detection kit of the present invention illustratively includes one or more types of HBoV VLPs; and one or more ancillary reagents such as a high binding microtiter plate or other support, blocking agent, washing buffer such as phosphate buffered saline, a labeled anti-immunoglobulin antibody, and matching detection agents, swab or other sample collection devices, control reagents such as labeled non-competing or unlabelled reagents, control nucleotide sequence and relevant primers and probes, and other materials and reagents for detection. The kit optionally includes instructions printed or in electronically accessible form and/or customer support contact information.

Anti-immunoglobulin antibodies in a signal producing system or otherwise are optionally labeled with a fluorophore, biotin, peroxidase, or other enzymatic or non-enzymatic detection label. It is appreciated that a signal producing system may employ an unlabeled primary antibody and a labeled secondary antibody derived from the same or a different organism. It is further appreciated that non-antibody signal producing systems are similarly operable.

It is further appreciated that a kit optionally includes ancillary reagents such as buffers, solvents, a detectable label and other reagents necessary and recognized in the art for detection of an antibody in a biological sample.

Optionally, a kit of the present invention contains reagents for PCR based detection of HBoV genes, either structural or non-structural.

### Pharmaceutical Compositions and Processes

Vaccines and methods for their use to induce active immunity and protection against HBoV-induced illness in a subject are provided according to the present invention.

In particular embodiments, HBoV VLPs are administered as antigens for prevention or treatment of HBoV infection such as by serving as an active vaccine component, or by eliciting an immune response in a host organism. Vaccine delivery may occur prior to or following HBoV infection of a host organism or patient. A vaccine optionally contains one or more adjuvants and preservatives or other pharmaceutically acceptable carrier.

In particular embodiments, vaccine compositions include one or more types of HBoV VLP admixed with a pharmaceutically acceptable carrier.

The term “pharmaceutically acceptable carrier” refers to a carrier which is substantially non-toxic to a subject and substantially inert to the HBoV VLPs included in a vaccine composition. A pharmaceutically acceptable carrier is a solid, liquid or gel in form and is typically sterile and pyrogen free. An adjuvant is optionally included in a virus composition according to embodiments of the present invention. Adjuvants are known in the art and illustratively include Freund's adjuvant, aluminum hydroxide, aluminum phosphate, aluminum oxide, saponin, dextrans such as DEAE-dextran, vegetable oils such as peanut oil, olive oil, and/or vitamin E acetate, mineral oil, bacterial lipopolysaccharides, peptidoglycans, and proteoglycans.

Detailed information concerning customary ingredients, equipment and processes for preparing dosage forms is found in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 20th ed., 2003; and J. G. Hardman et al., Goodman & Gilman's The Pharmacological Basis of Therapeutics, McGraw-Hill Professional, 10th ed., 2001.

A vaccine composition of the present invention may be in any form suitable for administration to a subject.

A vaccine composition is administered by any suitable route of administration including oral and parenteral such as intravenous, intradermal, intramuscular, intraperitoneal, mucosal, nasal, or subcutaneous routes of administration.

The phrase “therapeutically effective amount” refers to an amount effective to induce an immunological response and prevent or ameliorate signs or symptoms of HBoV-mediated disease. Induction of an immunological response in a subject can be determined by any of various techniques known in the art, illustratively including detection of anti-HBoV antibodies, measurement of anti-HBoV antibody titer and/or lymphocyte proliferation assay. Signs and symptoms of HBoV-mediated disease may be monitored to detect induction of an immunological response to administration of a vaccine composition of the present invention in a subject.

Administration of a vaccine composition according to a method of the present invention includes administration of one or more doses of a vaccine composition to a subject at one time in particular embodiments. Alternatively, two or more doses of a vaccine composition are administered at time intervals of weeks-years. A suitable schedule for administration of vaccine composition doses depends on several factors including age and health status of the subject, type of vaccine composition used and route of administration, for example. One of skill in the art is able to readily determine a dose and schedule of administration to be administered to a particular subject.

Immunogenicity of HBoV VLPs is tested by any of various assays known in the art. In a particular example, purified HBoV VLPs are administered intramuscularly to mice with or without an adjuvant. Immunogenicity is assayed by measuring immunoglobulin titers including IgM, IgA and/or IgG in blood samples obtained at various times after administration.

Neutralizing antibody titers are measured by neutralization assays known in the art, such as those generally described in Kuby, J., Immunology, 3rd ed. W.H. Freeman and Co., New York, N.Y., 1997. For example, sera from mice injected with HBoV VLPs are serially diluted two-fold in duplicate wells and incubated with trypsin-inactivated HBoV. Active HBoV or serum-free MEM medium is incubated in the absence of mouse serum and serve as positive and negative controls, respectively. HeLa cells in MEM medium supplemented with 0.5% calf serum are added to each well. After incubation at 37° C. for 18 hours, cells are fixed with formalin. HBoV antigens in the fixed HeLa cells are detected by incubating cells with mouse anti-HBoV VP2, HRP-labeled anti-mouse IgG, and then tetramethyl benzidine. Neutralizing antibody titer in a serum is defined as the reciprocal of the highest dilution giving a 70% reduction in absorbance value compared to that in the virus control.

Optionally, antibodies raised to immunogenic HBoV VLPs are administered to a subject for prevention or therapeutic treatment relating to HBoV-mediated disease.

Additional therapeutics that are optionally administered with the vaccine composition or antibodies raised to HBoV VLPs include antivirals such as amantadine, rimantadine, gancyclovir, acyclovir, ribavirin, penciclovir, oseltamivir, foscarnet zidovudine (AZT), didanosine (ddI), lamivudine (3TC), zalcitabine (ddC), stavudine (d4T), nevirapine, delavirdine, indinavir, ritonavir, vidarabine, nelfinavir, saquinavir, relenza, tamiflu, pleconaril, interferons; steroids and corticosteroids such as prednisone, cortisone, fluticasone and glucocorticoid; antibiotics; analgesics; bronchodialaters; or other treatments for respiratory infection.

The invention also provides a pharmaceutical kit includes one or more receptacles containing one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In a preferred embodiment, the kit contains an antibody specific for HBoV VP2, HBoV VP1, the polypeptide of SEQ ID NO:1, an epitope or a variant thereof, or any HBoV epitope, a polypeptide or protein of the present invention, or a nucleic acid molecule of the invention, alone or in combination with adjuvants, antivirals, antibiotics, analgesic, bronchodialaters, or other pharmaceutically acceptable excipients. The present invention further encompasses kits comprising a container containing a pharmaceutical composition of the present invention and instructions for use.

Also provided is a diagnostic kit for detecting HBoV infection that contains HBoV VLPs as reagents for the detection of HBoV antibodies. It is further appreciated that a diagnostic kit optionally includes ancillary reagents such as buffers, solvents, a detectable label and other reagents necessary and recognized in the art for detection of an antibody in a biological sample.

### HBoV VLPs Containing a Cargo

Optionally, the VLP contains a cargo in the internal space defined by the VLP. In particular embodiments, a cargo moiety is a substance to be delivered to a subject or cell. Exemplary cargo moieties include an antigen, a nucleic acid which is not an intact HBoV genome and a therapeutic agent.

Particularly provided is a process of delivery of genetic information whereby genetic material is encapsulated in an HBoV capsid which is then introduced into a host cell. The genetic material is optionally DNA or RNA, or modifications thereof. The genetic information is optionally derived from an HBoV or other viral or nonviral organism, or is synthetic.

HBoV VLPs are used as antigens for production of monoclonal or polyclonal antibodies to HBoV for clinical use such as in therapy, analysis or diagnosis; or laboratory research.

A cargo is incorporated in the internal space defined by an HBoV VLP by any of various methods including introducing the cargo into a host cell such that HBoV VLPs are produced in the presence of the cargo and thereby include the cargo in the internal space. Alternatively or additionally, a cargo is incorporated in the internal space by incubating produced HBoV VLPs with the cargo such that the cargo enters the internal space, e.g. by diffusion.

### Anti-HBoV VLP Antibodies

In a preferred embodiment, HBoV VLPs are used for eliciting HBoV specific antibody or T cell responses to the VP1, VP2 or any antigen included therewith in the HBoV VLPs, in vivo (e.g., for protective or therapeutic purposes or for providing diagnostic antibodies) and in vitro (e.g., by phage display technology or another technique useful for generating synthetic antibodies).

HBoV-specific antibodies are provided according to the present invention which specifically bind to HBoV and do not specifically bind to other respiratory viruses, including adenovirus, influenza A, influenza B, respiratory syncytial virus (RSV), parainfluenza 1, parainfluenza 2 and parainfluenza 3.

A hybridoma cell line expressing monoclonal antibody raised against HBoV VLPs of the present invention, designated as Boca 2D1:1 E8, specifically binds to HBoV and does not specifically bind to other respiratory viruses, including adenovirus, influenza A, influenza B, respiratory syncytial virus (RSV), parainfluenza 1, parainfluenza 2 and parainfluenza 3.

An antibody raised to HBoV VLPs by any of the methods known in the art, is optionally purified by any method known in the art for purification of an immunoglobulin molecule, for example, by ion exchange chromatography, affinity, particularly by affinity for the specific antigen or size exclusion; centrifugation; differential solubility; or by any other standard techniques for the purification of proteins. It is also appreciated that an inventive antibody or fragments thereof may be fused to heterologous polypeptide sequences known in the art to facilitate purification.

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a constant region derived from a human immunoglobulin. Methods for producing chimeric antibodies are known in the art. (Morrison, 1985, Science, 229:1202; U.S. Pat. Nos. 5,807,715; 4,816,567; and 4,816,397). Humanized antibodies are antibody molecules from non-human species that bind the desired antigen having one or more complementarity determining regions (CDRs) from the non-human species and framework regions from a human immunoglobulin molecule. Often, framework residues in the human framework regions are substituted with the corresponding residue from the CDR donor antibody to alter, preferably improve, antigen binding. These framework substitutions are identified by methods well known in the art, such as by modeling of the interactions of the CDR and framework residues to identify framework residues important for antigen binding and sequence comparison to identify unusual framework residues at particular positions. (U.S. Pat. No. 5,585,089; Riechmann et al., 1988, Nature 332:323). Antibodies can be humanized using a variety of techniques known in the art including, for example, CDR-grafting (PCT publication WO 91/09967; U.S. Pat. Nos. 5,225,539; 5,530,101 and 5,585,089), veneering or resurfacing (Studnicka et al., 1994, Protein Engineering 7(6):805 814; Roguska et al., 1994, PNAS. 91:969 973), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. (U.S. Pat. Nos. 4,444,887 and 4,716,111).

Human antibodies are readily produced using transgenic mice which are incapable of expressing functional endogenous immunoglobulins, but which can express human immunoglobulin genes and are produced to order by Medarex or Genpharm.

An inventive antibody is optionally fused or conjugated to heterologous polypeptides may be used in vitro immunoassays and in purification methods such as affinity chromatography. (PCT publication Number WO 93/21232; U.S. Pat. No. 5,474,981).

An inventive antibody is optionally attached to solid supports, which are particularly useful for immunoassays or purification of the polypeptides of the invention or fragments, derivatives, analogs, or variants thereof, or similar molecules having the similar enzymatic activities as the polypeptide of the invention. Such solid supports include, but are not limited to, glass, cellulose, polyacrylamide, nylon, polystyrene, polyvinyl chloride or polypropylene.

### Assays for HBoV

Anti-HBoV VLP antibodies of the present invention are used to detect HBoV in a biological sample in preferred embodiments of the present invention.

An assay for HBoV in a biological sample of the present invention includes contacting a biological sample with an anti-HBoV antibody and detecting formation of a complex between anti-HBoV antibody and the HBoV present in the biological sample. Formation of the complex is indicative of current infection by HBoV in a subject from which a biological sample is obtained. Formation of the complex specifically indicates presence of HBoV since other respiratory viruses, particularly parvovirus B19, do not form a complex with an anti-HBoV antibody of the present invention.

In a specific embodiment, the processes further involve obtaining a biological sample from a subject, contacting the sample with a compound or agent capable of detecting the presence of HBoV nucleic acid in the sample in order to confirm presence of HBoV in the sample.

In further embodiments, a control sample is assayed for presence of HBoV and/or anti-HBoV antibodies and results are compared with a test sample to ascertain a difference in presence or amount of HBoV or anti-HBoV antibodies.

The invention also encompasses kits for detecting the presence of HBoV in a test sample. The kit, for example, includes an anti-HBoV antibody and optionally includes a reagent such as a labeled secondary antibody or agent capable of detecting an antibody in a complex with an HBoV and, in certain embodiments, for determining the titer in the sample.

As used herein, the terms “subject” and “patient” are synonymous and refer to a non-human animal, preferably a mammal including a non-primate such as cows, pigs, horses, goats, sheep, cats, dogs, avian species and rodents; and a non-human primate such as monkeys, chimpanzees, and apes; and a human, also denoted specifically as a “human subject”.

The present invention is further illustrated with respect to the following non-limiting examples.

### Example 1

### HBoV DNA

HBoV DNA, obtained from a nasopharyngeal swab specimen, is detected according to real-time PCR assays previously described (Lu, 2006).

Baculovirus-expressed VP2 protein derived from HBoV.

HBoV VP2 sequence (SEQ ID Nos. 1 and 2) derived from the HBoV DNA has been deposited in GenBank (accession number EU078168). HBoV VP2 of SEQ ID No. 2 is amplified by hot-start PCR (Novagen KOD Hot Start DNA Polymerase, EMD Chemicals Inc, La Jolla, Calif.) per manufacturer's instructions. Primers flanked the HBoV VP2 gene and incorporated restriction sites for NotI and XbaI (lower case), respectively, HBoV_VP2_FW (5′ GAA CCT AAA Cgc ggc cgc TCA AAA ATG TCT 3′ SEQ ID No.3) and HBoV_VP1/VP2_RV (5′ CAA CG t cta ga A TAA AGA TTA CAA CAC TTT ATT 3′ SEQ ID No.4). Amplification conditions consisted of 2 min at 94° C., followed by 35 cycles (94° C./15 sec; 52° C./1 min; 72° C./2 min) and 10 min at 72° C. PCR products are purified from a low-melt agarose gel (QIAquick Gel Extraction Kit, Qiagen) and double-digested with NotI and XbaI (New England BioLabs, Ipswich, Mass.). Digested HBoV VP2 gene is cloned in frame into NotI-XbaI sites of pFastBac1 vector (Invitrogen Corp, Carlsbad, Calif.) originating the recombinant plasmid HBoV_VP2_pFastBac1. After confirmatory sequencing, HBoV_VP2_pFastBac1 is used as the donor plasmid to generate recombinant baculovirus expressing HBoV VP2 protein of SEQ ID No. 1 by site-specific transposon-mediated insertion (Luckow, 1993) using a commercial baculovirus system (Bac-to-Bac, Invitrogen Corp) per manufacturer's instructions.

HBoV VP1 sequence (SEQ ID No. 6) derived from the HBoV DNA is amplified by hot-start PCR (Novagen KOD Hot Start DNA Polymerase, EMD Chemicals Inc, La Jolla, Calif.) per manufacturer's instructions. Primers flanked the HBoV VP1 gene and incorporated restriction sites for NotI and XbaI (lower case), respectively, HBoV_VP1_FW (5′ GAA CCT AAA Cgc ggc cgc AAG CAG ATG CCT 3′ SEQ ID No.7) and (5′ CAA CGt cta gaA TAA AGA TTA CAA CAC TTT ATT 3′ SEQ ID No.4). Amplification conditions consisted of 2 min at 94° C., followed by 35 cycles (94° C./15 sec; 52° C./21 min; 72° C./2 min) and 10 min at 72° C. PCR products are purified from a low-melt agarose gel (QIAquick Gel Extraction Kit, Qiagen) and double-digested with NotI and XbaI (New England BioLabs, Ipswich, Mass.). Digested HBoV VP1 gene is cloned in frame into NotI-XbaI sites of pFastBac1 vector (Invitrogen Corp, Carlsbad, Calif.) originating the recombinant plasmid HBoV_VP1_pFastBac1. After confirmatory sequencing, HBoV_VP1_pFastBac1 is used as the donor plasmid to generate recombinant baculovirus expressing HBoV VP1 protein of SEQ ID No. 5 by site-specific transposon-mediated insertion (Luckow, 1993) using a commercial baculovirus system (Bac-to-Bac, Invitrogen Corp) per manufacturer's instructions.

### Example 2

### HBoV VLPs Generation and Characterization

Standard baculovirus techniques are used for generation and amplification of the recombinant baculovirus expressing the HBoV VP2 protein generated as described in Example 1. Spodoptera frugiperda insect cells (Sf9, ATCC CRL-1711) are used to generate the recombinant baculovirus. Briefly, 6-well plates containing 8×10⁶ cells/ml are infected with 1.5 ng of purified recombinant bacmid DNA and 7 microliters of Cellfectin (Invitrogen Corp) in serum-free media (HyQ SFX-Insect, Logan, Utah) and incubated at 27° C. After 72 hours, an aliquot of the cells are submitted to negative staining electron microscopy and immunofluorescence with a pool of HBoV positive human convalescent sera. Upon confirmation of expressed HBoV VLPs, the initial recombinant baculovirus is used as viral inoculum for a subsequent virus passage which is in turn submitted to plaque assay (titer 3.5×10e7 pfu/ml). One single plaque is used to generate a working stock used in the subsequent VLP preparations.

### Example 3

### Isolation of HBoV Virus-Like Particles

HBoV virus-like particles are isolated from Sf9 cells. Briefly, Sf9 cells are grown in 150-cm² flasks in Grace's medium with 5% FCS and antibiotics. Confluent cell cultures infected with VP1 or VP2 containing baculovirus are maintained for five days in supplemented Grace's medium and are then harvested by low speed centrifugation and suspension in Tris Buffered Saline. Lysates are generated by sonication in the presence of protease inhibitors followed by concentration of virus-like particles through 2 ml of a 2% sucrose shelf in Tris buffered saline and centrifugation and 35,000 RPM in a Beckman SW41 rotor. The isolated bands are treated by DNase I for 45 min at ambient temperature and then loaded onto a cesium chloride gradient for centrifugation and isolation as above. Further details of an isolation method are described in Gillock, E T. et al, 1997. J. Virol., 71:2857-2865, specifically pg. 2858.

### Example 4

### Electron Microscopy of HBoV Virus-Like Particles

CsCl-purified recombinant HBoV virus-like particles are placed on a pioloform-coated grid and allowed to adsorb for 5 min. After a distilled water rinse, the sample is stained with a 1% aqueous uranyl acetate solution and examined with a Philips 201 electron microscope operating at 60 kV.

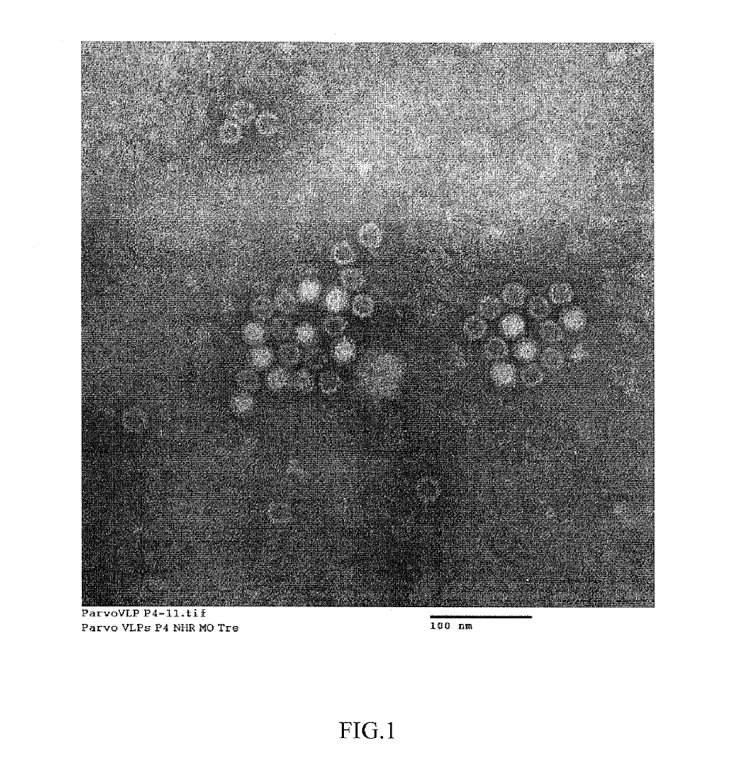

FIG. 1 shows an electron micrograph of HBoV virus-like particles obtained after Sf9 cells transfection with recombinant baculovirus containing the HBoV VP2 gene (72 hours p.i.).

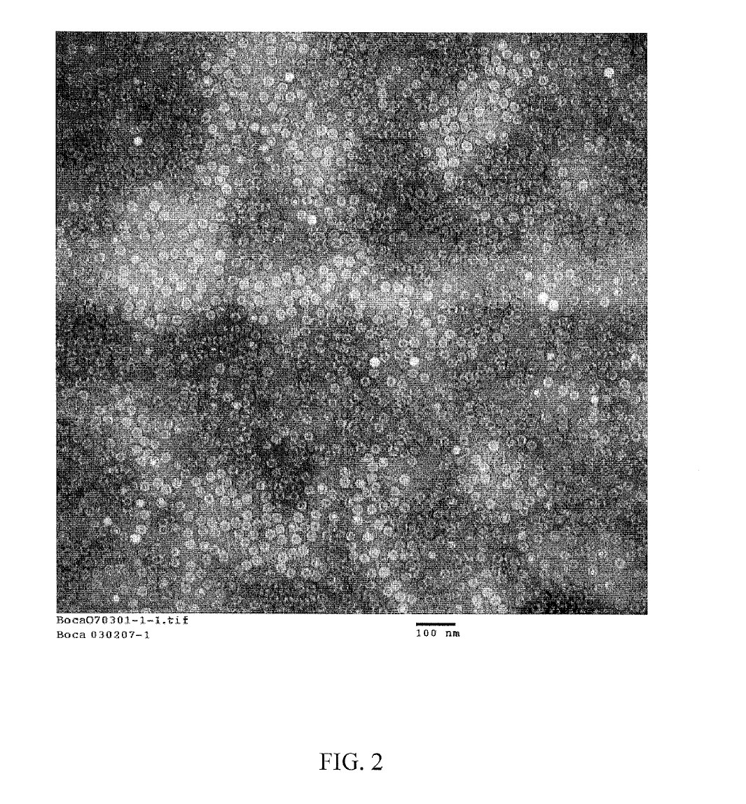

FIG. 2 shows an electron micrograph of HBoV virus-like particles obtained after High5 cell infection (mid-scale production) with recombinant baculovirus containing the HBoV VP2 gene (96 hours p.i.).

In further a example, HBoV VLPs are prepared for negative staining electron microscopy examination by use of 2% phosphotungstic acid negative staining. Formvar-carbon grids are pre-treated with glow discharge. Samples are mixed 1:1 with catalase crystals and prepared for negative-stain EM examination to determine the HBoV VLP dimensions and particle counts.

### Example 5

### Production of Anti-HBoV Mice Hyperimmune Sera and Monoclonal Antibodies

Female adult BALB/c mice are used according to an approved animal protocol. In brief, each mouse is immunized with 25 ug of purified HBoV VLPs mixed with equal volume of complete Freund's adjuvant (Sigma-Aldrich, St. Louis, Mo.). After two immunizations at days 7 and 14 with incomplete Freund's and no adjuvant, respectively, tail bleeds are obtained and sera tested by immunofluorescence. After 2 additional immunizations 14 days apart, one additional tail bleed is obtained, sera are tested and the mice are submitted to a final boost. Following anesthesia, mice are bled by heart puncture and blood and spleens are collected. Serum samples are tested, pooled and submitted to IgG affinity purification (NAb Protein A/G Spin Kit, Pierce Biotechnology, Rockford, Ill.). Purified antibody is dialyzed, retested and used as positive control during monoclonal antibody production. Spleen cells are harvested, fused with SP2/0 cells and resulting hybridomas are cloned three times by limiting dilution according to standard protocols.

### Example 6

Indirect immunofluorescence assay for detection of anti-HBoV antibodies (HBoV IFA). HBoV-infected and non-infected High 5 cells (5×10⁵ cells/mL) are applied to either 24-well slides or 96-well flat bottom plates. Cells are air dried, fixed in cold acetone/10 mM PBS 7.4 (80/20) for 5 min at −20° C., dried and stored at 4° C. for short-term and −70° C. for long-term storage. Human sera, mice sera or hybridoma supernatants are diluted in 5% milk, 0.15% Tween 20, 10 mM PBS pH 7.4 (PBS/M/T) and 2.5% BSA/10 mM PBS 7.4 (PBS/B), respectively, applied to the wells and incubated at 37° C. for 45 min. Slides or plates are washed 3 times with 0.01% Tween 20/10 mM PBS 7.4 and incubated with Alexa Fluor 488-conjugated goat anti-mouse IgG IgM (Molecular Probes, Invitrogen Corp) or fluorescein isothiocyanate goat anti-human IgA+IgG+IgM (H+ L) antibody (KPL Inc., Gaithersburg, Md.) at 37° C. for 45 min. After washing, mounted slides and 96-well plates are examined on an inverted fluorescent scope (Axiovert 200, Carl Zeiss Inc., Germany). Selected images are captured by use of an AxioVision image processing and analysis system (Carl Zeiss Inc.).

### Example 7

### Detection of HBoV Antibodies in Sera from'Human Patients

Archived human sera consisted of healthy adult blood donors, infants and reference B19 serology panel are used. Most of the serum samples have been previously submitted to the Respiratory Virus Diagnostic Program at the Centers for Disease Control and Prevention (CDC) for diagnostic testing. Sample subjects could not be identified or linked through identifiers.

Briefly, Sf9 cells infected with recombinant baculovirus expressing HBoV protein VP2 are grown in suspension are harvested 24-72 hours post-infection, washed, and resuspended in Grace's insect medium for 1 h at ambient temperature. Cells are pelleted on microscope slides at 600×g for 10 min using a cytospin centrifuge, fixed in 70% methanol, 30% acetic acid for 10 min at −20° C. followed by 5 min at room temperature, and then incubated with 5% normal goat serum in PBS for 30 min at room temperature. Cells are then incubated overnight at 4° C. with either human patient serum or nonspecific antibody. Uninfected cells are used as a control in a separate reaction. A rhodamine-conjugated goat anti-human is added at a dilution of 1:50 in PBS containing 5% normal goat serum for 1 h. Cells are washed three times, after each incubation with PBS. Cells are visualized using confocal microscopy.

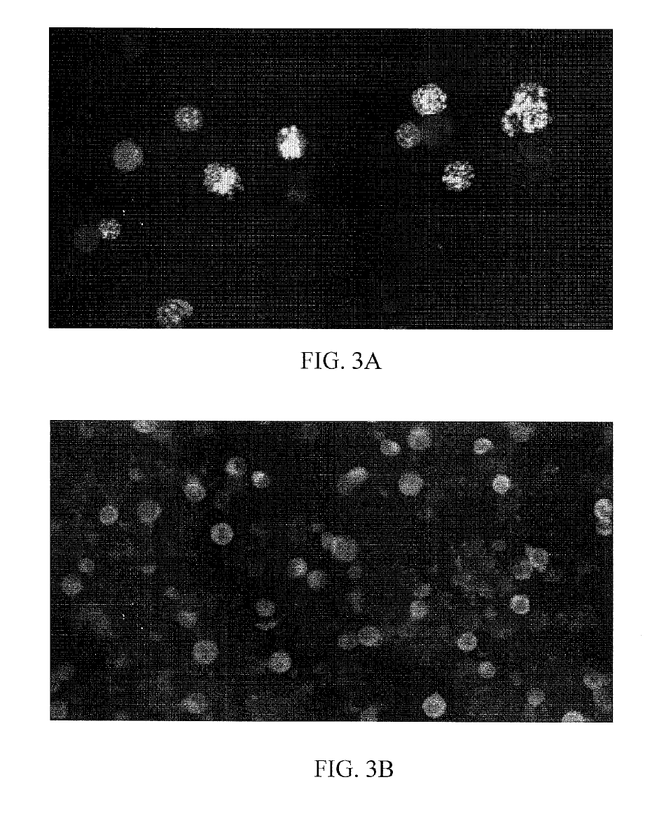

FIG. 3A shows positive results of this indirect immunofluorescence assay (IFA) using human sera from patients positive for HBoV incubated with SF-9 cells infected with recombinant baculovirus-expressed HBoV protein (VP2) (72 hours p.i.) and FIG. 3B shows that the control using human sera from patients positive for HBoV incubated with uninfected Sf9 cells (72 hours p.i.) is negative.

### Example 8

### Detection of HBoV Antibodies in Sera from HBoV Virus-Like Particle Immunized Mice

Immunofluorescence assays are performed using serum samples obtained from mice immunized with HBoV VLPs.

Sf9 cells infected with recombinant baculovirus expressing HBoV protein VP2 are grown in suspension are harvested 24-72 hours post-infection, washed, and resuspended in Grace's insect medium for 1 h at ambient temperature. Cells are pelleted on microscope slides at 600×g for 10 min using a cytospin centrifuge, fixed in 70% methanol, 30% acetic acid for 10 min at −20° C. followed by 5 min at room temperature, and then incubated with 5% normal goat serum in PBS for 30 min at room temperature. Cells are then incubated overnight at 4° C. with either HBoV virus-like particle immunized mouse serum or nonspecific antibody. Uninfected cells are used as a control in a separate reaction. A rhodamine-conjugated goat-anti mouse antibody is added at a dilution of 1:50 in PBS containing 5% normal goat serum for 1 h. Cells are washed three times, after each incubation with PBS. Cells are visualized using confocal microscopy.

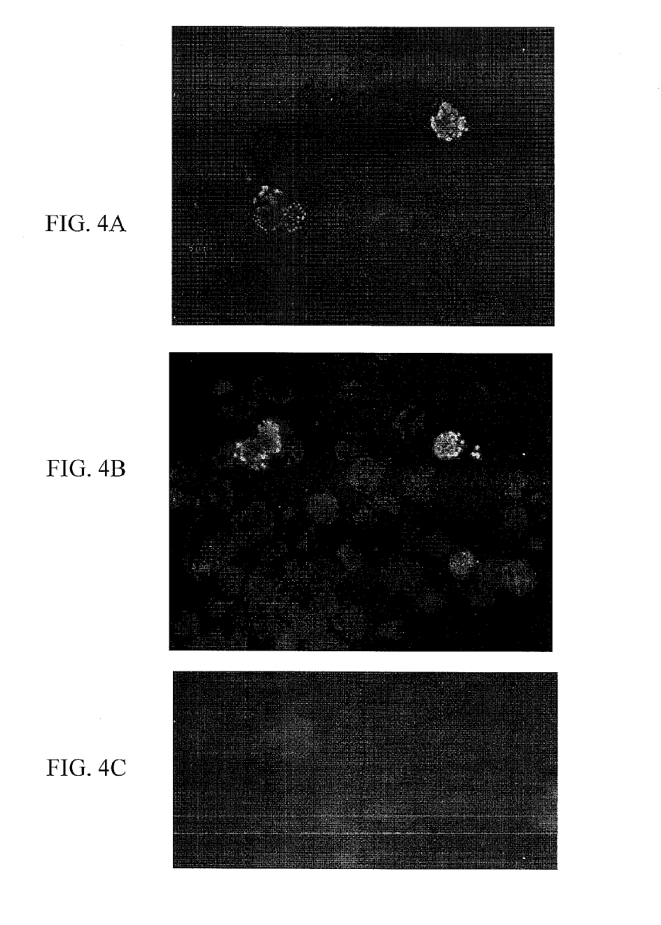

FIGS. 4A and 4B show positive results of this IFA using sera from mice immunized with purified HBoV VLPs incubated with SF-9 cells infected with recombinant baculovirus-expressed HBoV protein (VP2) (72 hours p.i.). FIG. 4C shows negative results in a control reaction using sera from mice immunized with purified HBoV VLPs incubated with uninfected Sf9 cells (72 hours p.i.).

### Example 9

### Monoclonal Antibodies of the Present Invention Specifically Recognize HBoV Virus-Like Particles

Baculovirus-infected Sf9 cells expressing HBoV virus-like particles are grown in suspension and are harvested 24-72 hours post-infection, washed, and resuspended in Grace's insect medium for 1 h at ambient temperature. Cells are pelleted on microscope slides at 600×g for 10 min using a cytospin centrifuge, fixed in 70% methanol, 30% acetic acid for 10 min at −20° C. followed by 5 min at room temperature, and then incubated with 5% normal goat serum in PBS for 30 min at room temperature. Cells are then incubated overnight at 4° C. with either HBoV specific monoclonal antibody or nonspecific antibody. Uninfected cells are used as a control in a separate reaction. A rhodamine-conjugated goat-anti mouse antibody is added at a dilution of 1:50 in PBS containing 5% normal goat serum for 1 h. Cells are washed three times, after each incubation with PBS. Cells are visualized using confocal microscopy.

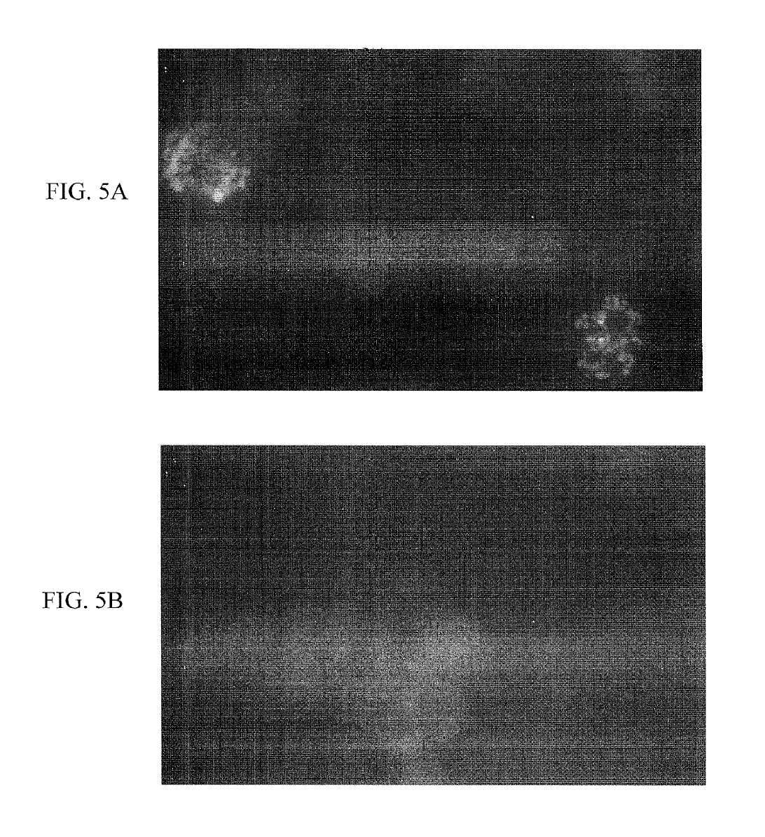

FIG. 5A shows positive results of an IFA using a monoclonal antibody to HBoV VLPs incubated with Sf9 cells infected with recombinant baculovirus-expressed HBoV protein (VP2) (72 hours p.i.). In contrast, FIG. 5B shows a negative control using a monoclonal antibody to HBoV VLPs incubated with uninfected Sf9 cells.

### Example 10

### Monoclonal Antibodies of the Present Invention Specifically Recognize HBoV and not Other Respiratory Viruses

A panel of respiratory virus specimens is assayed using monoclonal antibodies, Boca 2D1:1 E8; and Boca 2D4, to determine binding specificity. Commercial preparations of adenovirus, influenza A, influenza B, respiratory syncytial virus (RSV), parainfluenza 1, parainfluenza 2 and parainfluenza 3 are tested by immunofluorescence assay for binding of monoclonal Abs Boca 2D1:1 E8; and Boca 2D4. HBoV VLPs including HBoV VP2 and no HBoV VP1 of the present invention are used as a positive control. Commercially available antibodies specific for adenovirus, influenza A, influenza B, respiratory syncytial virus (RSV), parainfluenza 1, parainfluenza 2 and parainfluenza 3 are used to confirm presence of each type of virus in the specimens assayed.

Briefly described, specimens are incubated with each monoclonal anti-HBoV antibody, Boca 2D1:1 E8; and Boca 2D4. Following incubation the specimens are washed and then incubated with an FITC-labeled secondary antibody appropriate for detection of the anti-HBoV monoclonals or control antibodies. The specimens are washed and examined.

### Example 11

### Enzyme Immunoassay for Detection of HBoV Antibodies (HBoV VLP EIA)

HBoV VLPs are cross-titrated against archived sera to determine the optimal protein concentration. Microtiter plates (Immulon 2HB, Thermo Scientific, Waltham, Mass.) are coated with 1000 ng of VLPs produced as described in Example 2 diluted in 10 mM PBS pH 7.4 and incubated overnight at 4° C. Supernatant consisted of 1000 ng of uninfected High 5 cell lysate is used as negative control. After 3 washes with 10 mM PBS pH 7.4 and 0.05% Tween 20 (PBS/T), wells are blocked with 5% milk, 0.15% Tween 20, 10 mM PBS pH 7.4 (PBS/M/T) for 1 hour. Human sera diluted 1:100 or monoclonal supernatants are diluted in PBS/M/T, added to the plates and incubated for 1.5 h at 37° C. followed by 3 washes with PBS/T. Anti-human IgA IgG IgM (H+ L) peroxidase diluted 1:4,000 in PBS/M/T is added and incubated for 1 h at 37° C. Plates are washed with PBS/T and tetramethylbenzidine (TMB) substrate is added and incubated for 15 min at room temperature. The reaction is stopped by the addition of 2 M H₃PO₄, and absorbance is measured at 450 and 630 nm. The difference in the mean absorbance values (P−N) and the ratio of the mean absorbance values (P/N) of two antigen-positive (P) and two antigen-negative (N) control wells is calculated for each serum specimen. Positive and a negative serum controls are included in each assay, to ensure reproducibility of the results.

### Example 12

### Enzyme Immunoassay of Human Serum from HBoV Infected Patients

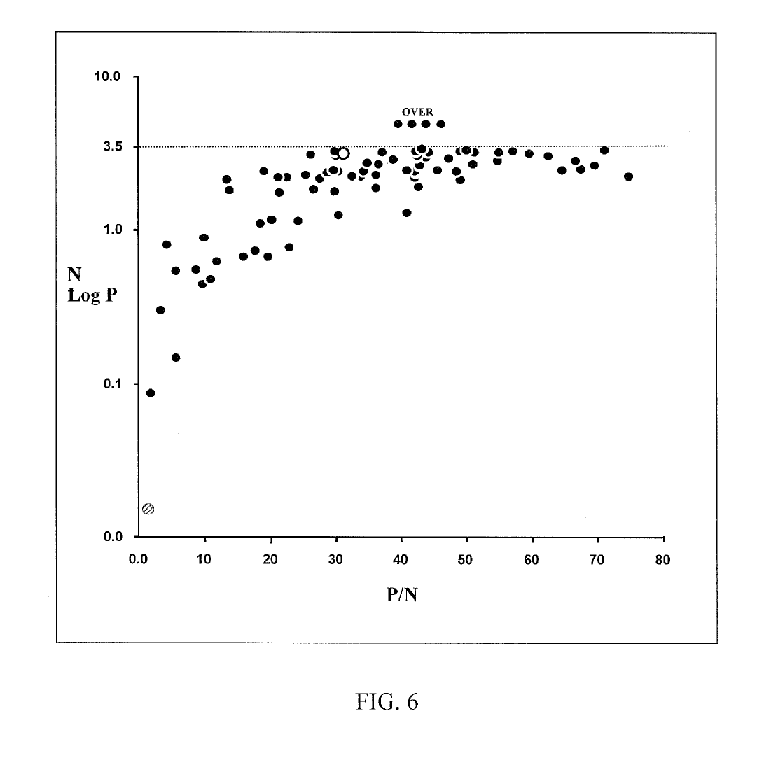

Isolated HBoV VLPs in phosphate buffered saline are used to coat high binding polystyrene 96-well plates overnight at 4° C. The material is removed from the wells of the plate and unoccupied binding sites are blocked with 100 microliters of blocking buffer containing 100 mM phosphate buffer, pH 7.2, 1% BSA, 0.5% Tween-20 and 0.02% Thimerosol for 30 min at ambient temperature. The solution is removed and the plate washed 3× in wash buffer (100 mM phosphate buffer, 150 mM NaCl, 0.2% BSA and 0.05% Tween 20). Human serum from eighty-one healthy adult blood donors and infants is diluted in wash buffer is added to individual wells and allowed to incubate for 1 hour at ambient temperature followed by removal of the solution and three washes with wash buffer. Peroxidase labeled anti-human IgG and IgM antibody in 0.1M Bicarbonate buffer, pH 9.2, is added to each well and incubated for 30 min at ambient temperature. The solution is aspirated and the wells washed 3× in wash buffer followed by development and detection on a spectrophotometer. Results are depicted in the graph of FIG. 6 which indicates that all sera tested are positive for HBoV antibodies except one sample from an infant (cross-hatched dot). A B19 IgM/IgG negative control serum also tested positive for HBoV antibodies (unfilled dot).

### Example 13

### Detection of HBoV in Human Sera by Immunoelectron Microscopy

CsCl-purified recombinant HBoV VLPs are incubated with serum from HBoV positive patients or negative serum followed by incubation with gold conjugated anti-human IgG antibody. The complexes are placed on a pioloform-coated grid and allowed to adsorb for 5 min. After a distilled water rinse, the sample is stained with a 1% aqueous uranyl acetate solution and examined with a Philips 201 electron microscope operating at 60 kV.

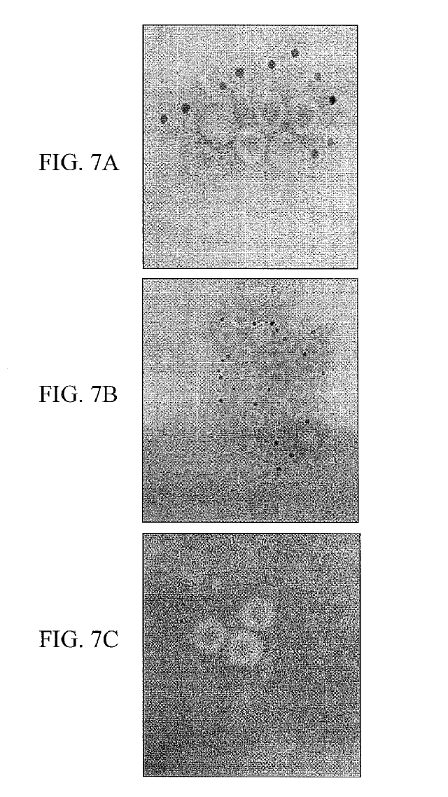

FIGS. 7A and 7B show electron micrographs including gold particles (dark spots) bound to HBoV VLPs indicating specific binding of the serum antibodies recognizing HBoV VLPs. The bar represents 100 nm. FIG. 7C shows a negative control electron micrograph having no gold particles bound to the HBoV VLPs following incubation with HBoV negative serum and an anti-human secondary antibody conjugated with gold particles. The bar represents 100 nm.

Nucleic Acid and Amino Acid Sequences

| SEQ ID No. 1 | |
|---|---|
| HBoV VP2 | |
| MSDTDIQDQQPDTVDAPQNTSGGGTGSIGGGKGSGVGISTGGWVGGSHFSDKYVVTK | |
| NTRQFITTIQNGHLYKTEAIETTNQSGKSQRCVTTPWTYFNFNQYSCHFSPQDWQRLTN | |
| EYKRFRPKAMQVKIYNLQIKQILSNGADTTYNNDLTAGVHIFCDGEHAYPNASHPWDE | |
| DVMPDLPYKTWKLFQYGYIPIENELADLDGNAAGGNATEKALLYQMPFFLLENSDHQ | |
| VLRTGESTEFTFNFDCEWVNNERAYIPPGLMFNPKVPTRRVQYIRQNGSTAASTGRIQP | |
| YSKPTSWMTGPGLLSAQRVGPQSSDTAPFMVCTNPEGTHINTGAAGFGSGFDPPSGCLA | |
| PTNLEYKLQWYQTPEGTGNNGNIIANPSLSMLRDQLLYKGNQTTYNLVGDIWMFPNQV | |
| WDRFPITRENPIWCKKPRADKHTIMDPFDGSIAMDHPPGTIFIKMAKIPVPTATNADSYL | |
| NIYCTGQVSCEIVWEVERYATKNWRPERRHTALGMSLGGESNYTPTYHVDPTGAYIQP | |
| TSYDQCMPVKTNINKVL | |
| SEQ ID No. 2 | |
| cDNA encoding HBoV VP2 1629 bp | |
| 1 | atgtctgaca ctgacattca agaccaacaa cctgatactg tggacgcacc acagaacacc |
| 61 | tcagggggag gaacaggaag tattggagga ggaaaaggat ctggtgtggg gatttccact |
| 121 | ggagggtggg tcggaggttc tcacttttca gacaaatatg tggttactaa aaacacaaga |
| 181 | caatttataa ccacaattca gaatggtcac ctctacaaaa cagaggccat tgaaacaaca |
| 241 | aaccaaagtg gaaaatcaca gcgctgcgtc acaactccat ggacatactt taactttaat |
| 301 | caatacagct gtcacttctc accacaagat tggcagcgcc ttacaaatga atataagcgc |
| 361 | ttcagaccta aagcaatgca agtaaagatt tacaacttgc aaataaaaca aatactttca |
| 421 | aatggtgctg acacaacata caacaatgac ctcacagctg gcgttcacat cttttgtgat |
| 481 | ggagagcatg cttacccaaa tgcatctcat ccatgggatg aggacgtcat gcctgatctt |
| 541 | ccatacaaga cctggaaact ttttcaatat ggatatattc ctattgaaaa tgaactcgca |
| 601 | gatcttgatg gaaatgcagc tggaggcaat gctacagaaa aagcacttct gtatcagatg |
| 661 | cctttttttc tacttgaaaa cagtgaccac caagtactta gaactggtga gagcactgaa |
| 721 | tttactttta actttgactg tgaatgggtt aataatgaaa gagcatacat tcctcctgga |
| 781 | ttgatgttca atccaaaagt tccaacaaga agagttcagt acataagaca aaacggaagc |
| 841 | acagcagcca gcacaggcag aattcagcca tactcaaaac caacaagctg gatgacagga |
| 901 | cctggcctgc tcagtgcaca gagagtagga ccacagtcat cagacactgc tccattcatg |
| 961 | gtttgcacta acccagaagg aacacacata aacacaggtg ctgcaggatt tggatctggc |
| 1021 | tttgatcctc caagcggatg tctggcacca actaacctag aatacaaact tcagtggtac |
| 1081 | cagacaccag aaggaacagg aaataatgga aacataattg caaacccatc actctcaatg |
| 1141 | cttagagacc aactcctata caaaggaaac cagaccacat acaatctagt gggggacata |
| 1201 | tggatgtttc caaatcaagt ctgggacaga tttcctatca ccagagaaaa tccaatctgg |
| 1261 | tgcaaaaaac caagggctga caaacacaca atcatggatc catttgatgg atccattgca |
| 1321 | atggatcatc ctccaggcac tatttttata aaaatggcaa aaattccagt accaactgca |
| 1381 | acaaatgcag actcatatct aaacatatac tgtactggac aagtcagctg tgaaattgta |
| 1441 | tgggaagtag aaagatacgc aacaaagaac tggcgtccag aaagaagaca tactgcactc |
| 1501 | gggatgtcac tgggaggaga gagcaactac acgcctacat accacgtgga tccaacagga |
| 1561 | gcatacatcc agcccacgtc atatgatcag tgtatgccag taaaaacaaa catcaataaa |
| 1621 | gtgttgtaa |
| SEQ ID No. 3 | |
| HBoV_VP2_FW | |
| 5′ GAA CCT AAA CGC GGC CGC TCA AAA ATG TCT 3′ | |
| SEQ ID No. 4 | |
| HBoV_VP1/VP2_RV | |
| 5′ CAA CGT CTA GAA TAA AGA TTA CAA CAC TTT ATT 3′ | |
| SEQ ID No. 5 | |
| HBoV VP1 | |
| MPPIKRQPRGWVLPGYRYLGPFNPLDNGEPVNNADRAAQLHDHAYSELIKSGKNPYLY | |
| FNKADEKFIDDLKDDWSIGGIIGSSFFKIKRAVAPALGNKERAQKRHFYFANSNKGAKK | |
| TKKSEPKPGTSKMSDTDIQDQQPDTVDAPQNTSGGGTGSIGGGKGSGVGISTGGWVGG | |
| SHFSDKYVVTKNTRQFITTIQNGHLYKTEAIETTNQSGKSQRCVTTPWTYFNFNQYSCH | |
| FSPQDWQRLTNEYKRFRPKAMQVKIYNLQIKQILSNGADTTYNNDLTAGVHIFCDGEH | |
| AYPNASHPWDEDVMPDLPYKTWKLFQYGYIPIENELADLDGNAAGGNATEKALLYQM | |
| PFFLLENSDHQVLRTGESTEFTFNFDCEWVNNERAYIPPGLMFNPKVPTRRVQYIRQNG | |
| STAASTGRIQPYSKPTSWMTGPGLLSAQRVGPQSSDTAPFMVCTNPEGTHINTGAAGFG | |
| SGFDPPSGCLAPTNLEYKLQWYQTPEGTGNNGNIIANPSLSMLRDQLLYKGNQTTYNL | |
| VGDIWMFPNQVWDRFPITRENPIWCKKPRADKHTIMDPFDGSIAMDHPPGTIFIKMAKIP | |
| VPTATNADSYLNIYCTGQVSCEIVWEVERYATKNWRPERRHTALGMSLGGESNYTPTY | |
| HVDPTGAYIQPTSYDQCMPVKTNINKVL | |
| SEQ ID No. 6 | |
| DNA encoding HBoV VP1 2035 bp | |
| 1 | atgcctccaa ttaagagaca gcctagaggg tgggtgctgc ctggatacag atatcttggg |
| 61 | ccatttaatc cacttgataa cggtgaacct gtaaataacg ctgatcgcgc tgctcaatta |
| 121 | catgatcacg cctactctga actaataaag agtggtaaaa atccatacct gtatttcaat |
| 181 | aaagctgatg aaaaattcat tgatgatcta aaagacgatt ggtcaattgg tggaattatt |
| 241 | ggatccagtt tttttaaaat aaagcgcgcc gtggctcctg ctctgggaaa taaagagaga |
| 301 | gcccaaaaaa gacactttta ctttgctaac tcaaataaag gtgcaaaaaa aacaaaaaaa |
| 361 | agtgaaccta aaccaggaac ctcaaaaatg tctgacactg acattcaaga ccaacaacct |
| 421 | gatactgtgg acgcaccaca gaacacctca gggggaggaa caggaagtat tggaggagga |
| 481 | aaaggatctg gtgtggggat ttccactgga gggtgggtcg gaggttctca cttttcagac |
| 541 | aaatatgtgg ttactaaaaa cacaagacaa tttataacca caattcagaa tggtcacctc |
| 601 | tacaaaacag aggccattga aacaacaaac caaagtggaa aatcacagcg ctgcgtcaca |
| 661 | actccatgga catactttaa ctttaatcaa tacagctgtc acttctcacc acaagattgg |
| 721 | cagcgcctta caaatgaata taagcgcttc agacctaaag caatgcaagt aaagatttac |
| 781 | aacttgcaaa taaaacaaat actttcaaat ggtgctgaca caacatacaa caatgacctc |
| 841 | acagctggcg ttcacatctt ttgtgatgga gagcatgctt acccaaatgc atctcatcca |
| 901 | tgggatgagg acgtcatgcc tgatcttcca tacaagacct ggaaactttt tcaatatgga |
| 961 | tatattccta ttgaaaatga actcgcagat cttgatggaa atgcagctgg aggcaatgct |
| 1021 | acagaaaaag cacttctgta tcagatgcct ttttttctac ttgaaaacag tgaccaccaa |
| 1081 | gtacttagaa ctggtgagag cactgaattt acttttaact ttgactgtga atgggttaat |
| 1141 | aatgaaagag catacattcc tcctggattg atgttcaatc caaaagttcc aacaagaaga |
| 1201 | gttcagtaca taagacaaaa cggaagcaca gcagccagca caggcagaat tcagccatac |
| 1261 | tcaaaaccaa caagctggat gacaggacct ggcctgctca gtgcacagag agtaggacca |
| 1321 | cagtcatcag acactgctcc attcatggtt tgcactaacc cagaaggaac acacataaac |
| 1381 | acaggtgctg caggatttgg atctggcttt gatcctccaa gcggatgtct ggcaccaact |
| 1441 | aacctagaat acaaacttca gtggtaccag acaccagaag gaacaggaaa taatggaaac |
| 1501 | ataattgcaa acccatcact ctcaatgctt agagaccaac tcctatacaa aggaaaccag |
| 1561 | accacataca atctagtggg ggacatatgg atgtttccaa atcaagtctg ggacagattt |
| 1621 | cctatcacca gagaaaatcc aatctggtgc aaaaaaccaa gggctgacaa acacacaatc |
| 1681 | atggatccat ttgatggatc cattgcaatg gatcatcctc caggcactat ttttataaaa |
| 1741 | atggcaaaaa ttccagtacc aactgcaaca aatgcagact catatctaaa catatactgt |
| 1801 | actggacaag tcagctgtga aattgtatgg gaagtagaaa gatacgcaac aaagaactgg |
| 1861 | cgtccagaaa gaagacatac tgcactcggg atgtcactgg gaggagagag caactacacg |
| 1921 | cctacatacc acgtggatcc aacaggagca tacatccagc ccacgtcata tgatcagtgt |
| 1981 | atgccagtaa aaacaaacat caataaagtg ttgtaatctt ataagcctct ttttt |

SEQ ID No. 7
GAA CCT AAA CGC GGC CGC AAG CAG ATG CCT

References cited or otherwise present herein are indicative of the level of skill in the art to which the invention pertains. These references are hereby incorporated by reference to the same extent as if each individual reference is explicitly and individually incorporated in full and individual text herein. U.S. Provisional Patent Application Ser. No. 61/069,470, filed Mar. 14, 2008, is hereby incorporated herein by reference in its entirety.

The compositions, methods and kits described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

### REFERENCE LIST

1. Allander, T., M. Tammi, M. Eriksson, A. Bjerkner, A. Tiveljung-Lindell, and B. Andersson. 2005. Cloning of a human parvovirus by molecular screening of respiratory tract samples. PNAS 102:12891-12896.http://jcm.asm.org/cgi/ijlink?linkType=ABST&journalCode=pnas&resid=102/36/12891
2. Bastien, N., K. Brandt, K. Dust, D. Ward, and Y. Li. 2006. Human bocavirus infection, Canada. Emerg. Infect. Dis. 12:848-850.
3. Bustin, S. A., and T. Nolan. 2004. A-Z of quantitative PCR. International University Line, La Jolla, Calif.
4. Chema, R., H. Sugawara, T. Koike, R. Lopez, T. J. Gibson, D. G. Higgins, and J. D. Thompson. 2003. Multiple sequence alignment with the Clustal series of programs. Nucleic Acids Res. 31:3497-3500.
5. Emery, S. L., D. D. Erdman, M. D. Bowen, B. R. Newton, J. M. Winchell, R. F. Meyer, S. Tong, B. T. Cook, B. P. Holloway, K. A. McCaustland, P. A. Rota, B. Bankamp, L. E. Lowe, T. G. Ksiazek, W. J. Bellini, and L. J. Anderson. 2004. Real-time reverse transcription-polymerase chain reaction assay for SARS-associated coronavirus. Emerg. Infect. Dis. 10:311-316.
6. Foulongne, V., M. Rodiere, and M. Segondy. 2006. Human bocavirus in children. Emerg. Infect. Dis. 12:862-863.
7. Ma, X., R. Endo, N. Ishiguro, T. Ebihara, H. Ishiko, T. Ariga, and H. Kikuta. 2006. Detection of human bocavirus in Japanese children with lower respiratory tract infections. J. Clin. Microbiol. 44:1132-1134.
8. Sloots, T. P., P. McErlean, D. J. Speicher, K. E. Arden, M. D. Nissen, and I. M. Mackay. 2006. Evidence of human coronavirus HKU1 and human bocavirus in Australian children. J. Clin. Virol. 35:99-102.
9. Tattersall, P. 2005. Family Parvoviridae, p. 353-369. In C. M. Fauquet, M. A. Mayo, J. Maniloff, U. Desselberger, and L. A. Ball (ed.), Virus taxonomy: classification and nomenclature of viruses. Eighth report of the International Committee on the Taxonomy of Viruses. Elsevier Academic Press, London, United Kingdom.
10. Xiaoyan Lu, Malinee Chittaganpitch, Sonja J. Olsen, Ian M. Mackay, Theo P. Sloots, Alicia M. Fry, and Dean D. Erdman. 2006. Real-Time PCR Assays for Detection of Bocavirus in Human Specimens. J. Clin. Microbiol., Vol. 44, p. 3231-3235.

1. A process of producing non-replicating, antigenic, human bocavirus virus-like particles comprising:
introducing into a host cell a first recombinant expression vector comprising a DNA sequence encoding at least one structural protein of human bocavirus capsid;
culturing the host cell under conditions such that the structural protein is produced and self assembles to form human bocavirus virus-like particles defining an internal space, with the proviso that the internal space contains no intact human bocavirus genome; and
isolating the human bocavirus virus-like particles.

2. The process according to claim 1, wherein the least one structural protein of human bocavirus capsid is human bocavirus VP2.

3. The process according to claim 1, wherein the least one structural protein of human bocavirus capsid is human bocavirus VP1 and human bocavirus VP2.

4. The process according to claim 1, wherein the recombinant expression vector is a baculovirus.

5. The process of claim 2, further comprising introducing into the host cell a second recombinant expression vector comprising a DNA sequence encoding at least human bocavirus VP1.

6. An isolated non-replicating, antigenic, human bocavirus virus-like particle comprising at least one structural protein of human bocavirus capsid.

7. The non-replicating, antigenic, human bocavirus virus-like particle of claim 6 comprising human bocavirus VP2 and substantially free of human bocavirus VP1.

8. The non-replicating, antigenic, human bocavirus virus-like particle of claim 7 comprising two structural proteins of human bocavirus capsid, VP1 and VP2, wherein the ratio of amounts of VP1 and VP2 in the virus-like particle is greater than the ratio of amounts of VP1 and VP2 in naturally occurring human bocavirus.

9. The non-replicating, antigenic, human bocavirus virus-like particle of claim 7 comprising two structural proteins of human bocavirus capsid, VP1 and VP2, in a ratio in the range of about 0.2:1-1:1, inclusive.

10. A process for detection of a human bocavirus antibody in a biological sample comprising:
contacting a first biological sample with a plurality of non-replicating, antigenic, human bocavirus virus-like particles according to claim 6; and
detecting the formation of a complex between an anti-human bocavirus antibody present in the first biological sample and the plurality of human bocavirus virus-like particles, to obtain a first signal indicative of the presence of an anti-human bocavirus antibody.

11. The process for detection of a human bocavirus antibody in a biological sample of claim 10, wherein the anti-human bocavirus antibody is an IgM antibody.

12. The process for detection of a human bocavirus antibody in a biological sample of claim 10, wherein the first biological sample is obtained from a subject in an acute phase of a viral disease; and further comprising:
contacting a second biological sample with a plurality of non-replicating, antigenic, human bocavirus virus-like particles according to claim 6, wherein the second sample is obtained from the subject in a convalescent phase of a viral disease;
detecting the formation of a complex between an anti-human bocavirus antibody present in the second biological sample and the human bocavirus virus-like particles to obtain a second signal indicative of the presence of an anti-human bocavirus antibody; and
comparing the first signal and second signal to detect a different amount of an anti-human bocavirus antibody present in the second biological sample compared to the first biological sample.

13-16. (canceled)

17. The process for detection of a human bocavirus antibody in a biological sample of claim 10, wherein the virus-like particles are attached to a solid substrate.

18-19. (canceled)

20. The process of producing non-replicating, antigenic, human bocavirus virus-like particles of claim 1 wherein the first recombinant expression vector comprises a DNA segment encoding HBoV VP2 of SEQ ID No. 1.

21. The process of producing non-replicating, antigenic, human bocavirus virus-like particles of claim 1 wherein the first recombinant expression vector comprises a DNA segment encoding a protein having at least 95% identity to SEQ ID No. 1, a protein encoded by SEQ ID No. 2, or a protein encoded by a nucleic acid sequence substantially identical to SEQ ID No. 2.

22. The process of producing non-replicating, antigenic, human bocavirus virus-like particles of claim 1 wherein the first recombinant expression vector comprises a DNA segment encoding HBoV VP1 of SEQ ID No. 5, a protein having at least 95% identity to SEQ ID No. 5, a protein encoded by SEQ ID No. 6, or a protein encoded by a nucleic acid sequence substantially identical to SEQ ID No.

23. The process of producing non-replicating, antigenic, human bocavirus virus-like particles according to claim 4 wherein the baculovirus is Autographa california nuclear polyhedrosis virus.

24. The isolated non-replicating, antigenic, human bocavirus virus-like particle of claim 6 comprising a human bocavirus structural protein selected from VP1 and VP2 bonded to a non-human bocavirus protein.

25-29. (canceled)
